(19) United States
(12) Patent Application Publication
(10) Pub. No.: US 2003/0108958 A1
(43) Pub. Date: Jun. 12, 2003

(54) Biological activity of AK155

(76) Inventors: Rene de Waal Malefyt, Sunnyvale, CA (US); Marehalli L. Nagalakshmi, Santa Clara, CA (US); Kevin Moore, Palo Alto, CA (US); Helmut Fickenscher, Heidelberg (DE)

Correspondence Address: DNAX RESEARCH, INC., LEGAL DEPARTMENT, 901 CALIFORNIA AVENUE, PALO ALTO, CA, 94304, US

(21) Appl. No.: 10/186,180

(22) Filed: Jun. 27, 2002

Related U.S. Application Data
(60) Provisional application No. 60/345,690, filed on Jan. 3, 2002.
(60) Provisional application No. 60/302,176, filed on Jun. 28, 2001.

(51) Int. Cl. G01N 33/567; C07H 21/04; C12P 21/02; C12N 5/06; C07K 14/715

(52) U.S. Cl. 435/7.21; 435/69.1; 435/320.1; 435/325; 530/350; 536/23.2

(57) ABSTRACT

This invention relates to cells that express a recombinant AK155 receptor, methods for screening for agent that modulates the effects of an AK155 on an AK155 receptor, and for methods of treating disease using agents that modulate the interactions between an AK155 and an AK155 receptor.

This filing claims benefit from U.S. Provisional Patent Applications 60/345,690 filed Jan. 3, 2002, and No. 60/302,176, filed Jun. 28, 2001.

### FIELD OF THE INVENTION

The present invention pertains to compositions related to proteins that function, e.g., in controlling the activity of immune cells. In particular, it provides purified genes, proteins, antibodies, and related reagents useful, e.g., to regulate the activity of various immune cell types.

### BACKGROUND OF THE INVENTION

Inflammation represents a cascade of physiological and immunological reactions that nature has designed as the first cellular response to noxious stimuli in an effort to localize toxic materials or prevent tissue injury. Clinically, inflammation is a primary disease under acute conditions or is a manifestation of the underlying pathology of chronic disease, characterized by classic signs of redness, swelling, heat, pain, and loss of function.

Regardless of the etiology, most forms of inflammation are propagated as a result of the activation of the immune system. In human beings, the immune response is composed of two major mechanisms, cell-mediated immunity and humoral (antibody) immunity (Nossal (1987) N. Engl. J. Med. 316:1320-1325). Both of these responses have a high level of specificity directed to antigenic epitopes expressed on molecular components of infectious agents, foreign (transplant) or transformed (cancer) cells, or even autologous cells (autoimmunity). The T cell receptor of CD8⁺ T cells is specific for peptide-MHC class I complexes on the surface of antigen-presenting cells. Most antigen-presenting cells in the body also express MHC class II. The peptide component of the MHC class II complex is derived from endogenous proteins synthesized within cells (e.g., viral infection, malignant transformation, or transplant antigens). Exogenous proteins are expressed by the antigen presenting cells and bound within the groove of MHC class I peptides for presentation. The recognition and binding of the CD8⁺ T cell receptor to the peptide-MHC class I complex in concert with CD4⁺ T helper cell lymphokines results in generation of cytolytic T cells capable of direct target cell lysis if the target cells display the specific peptide-MHC complex on their surface.

The humoral immune response is mediated by B lymphocytes and their cell surface receptors (membrane immunoglobulins) that are able to recognize epitopes displayed on the surface of intact protein molecules. The generation of an antibody response requires the triggering of CD4⁺ helper T cells (as described above) with interaction of CD4⁺ T cells and their lymphokines with B cells whose immunoglobulin cell surface receptor has bound a protein antigen (Powrie and Coffman (1993) Trends Pharmacol. Sci. 14:164-168). If this coordinated response occurs, B cells proliferate, differentiate into plasma cells, and secrete antibody molecules able to bind epitopes on the surface of protein molecules.

Cell signaling molecules such as cytokines, interleukins and other immunomodulatory signaling molecules serve to regulate immune responses, angiogenesis, cell growth, and the immune system through their interactions with their cognate receptors such as the interleukin receptors. The interleukin receptors form a large family of ligand-activated receptors that when activated or inhibited can affect the immune system, angiogenesis, cancer cells, etc. These receptors are typically heterodimeric in nature and are comprised of a ligand binding subunit (an α subunit) and a β subunit (which often plays a role in downstream signaling). It is important to identify which immunomodulatory ligand (e.g., interleukin) binds to which particular receptor(s) to elucidate the function of the ligand in physiological processes. This knowledge is useful in the discovery and development of agents that modulate the ligand's effects on its receptor(s).

One such ligand is an interleukin, AK155, that bears a low (less than 30%) amino acid identity to human IL-10 (see e.g., U.S. Pat. No. 5,989,867; Knappe et al. (2000) J. Virol. 74:3881-3887; GenBank Accession Nos. AJ251551 and AJ251549, which are all herein incorporated by reference for all purposes, including how to clone and express AK155 molecules). Among the cell types that AK155 is expressed in are T lymphocytes, L24 (a Hodgkin's lymphoma cell line), certain B cell lines, and peripheral blood mononuclear cells (see e.g., Knappe et al.). To date, no cognate receptor has been identified for an AK155 interleukin. Thus, a need exists for the identification of a receptor for an AK155 and for screening assays to identify agents that can act as modulators (e.g., agonists, antagonists, etc.) of an AK155-AK155 receptor complex formation and of the effects of an AK155 on physiological processes. The present invention fulfills these and other needs.

### SUMMARY OF THE INVENTION

In one aspect, the invention provides for cells, that express an AK155 cytokine receptor. In one aspect, the invention provides for cells recombinantly altered to express an exogenous AK155 cytokine receptor comprised of α and β subunits. The amino acid sequence of the AK155 receptor subunit β is at least 75% identical to SEQ ID NO:1; and the amino acid sequence of the AK155 receptor subunit β is at least 75% identical to SEQ ID NO:2. The AK155 cytokine receptor, when expressed in Ba/F3 cells, binds to AK155 and stimulates binding of STAT3 to interferon-γ-activated-sequences. Interferon-γ is abbreviated as IFN-γ. In certain embodiments, the amino acid sequence of the AK155 receptor cc subunit is SEQ ID NO: 1. In other embodiments, the amino acid sequence of the AK155 receptor β subunit is SEQ ID NO:2.

In another aspect, the invention provides methods for identifying anti-inflammatory agents wherein the agent inhibits AK155 activation of an AK155 receptor, which is defined as having an amino acid sequence of its α subunit that is at least 75% homologous to SEQ ID NO:1; and the amino acid sequence of its β subunit is at least 75% homologous to SEQ ID NO:2. The methods comprises contacting AK155 in a solution containing the receptor complex and a compound suspected of inhibiting receptor activation induced by binding of AK155 to the receptor and detecting an inhibition of AK155 receptor activation that normally results from AK155 binding to an AK155 receptor. In certain embodiments, an AK155 receptor is expressed in a cell. In other embodiments the inhibitor is a ligand that is a competitive inhibitor of an AK155 binding to an AK155 receptor. In still other embodiments, the inhibitor is a receptor specific antibody. In other embodiments the inhibitor is an antibody that binds to AK155. In still other embodiments detection is by analysis of the expression of IL-8, ICAM-1, ICAM-2, or B7-H1. Detection can also be carried out by the analysis of the phosphorylation and/or translocation of the transcription factor STAT3 to the nucleus of a cell. In other embodiments detection is by analysis of the binding of an activated STAT3 or a STAT3-receptor complex to DNA at IFN-γ-activated-sequences.

In another aspect, the invention provides for methods of inhibiting inflammation in a patient suffering from inflammatory disease. The methods typically comprise the administration of an antagonist of the AK155-AK155 receptor complex in an amount effective to inhibit AK155 activated inflammation. In certain embodiments the antagonist is an AK155 receptor antibody. In other embodiments the antagonist is an antibody that binds to AK155. In other embodiments the antagonist is a ligand that is a competitive inhibitor of AK155 binding to its receptor.

The invention further contemplates a method for detecting binding of AK155 to cell surface glycosaminoglycans of a cell, comprising adding AK155 to a first cell, adding AK155 and heparin to a second cell, and comparing binding of AK155 to said first and second cells, where lower binding with added heparin signifies that binding of AK155 to cell surface glycosaminoglycans occurs without added heparin.

### Definitions

“AK155” refers to a polypeptide having a sequence that has greater than 70% amino acid sequence identity, preferably greater than 75%, 80%, 85%, 90%, or 95% amino acid sequence identity, to SEQ ID No. 9.

“AK155 receptor” refers to a polypeptide that is a comprised of two subunits, α and β, which each have sequences that show greater than about 65% amino acid sequence identity, preferably about 70%, 75%, 80%, 85%, 90%, or 95% amino acid sequence identity, to SEQ ID No. 1 and SEQ ID No. 2, respectively. The term “AK 155 receptor complex” a complex of the alpha and beta subunits of the AK155 receptor. The term AK155 receptor therefore refers to polymorphic variants, alleles, mutants, and interspecies homologs that: (1) have the characteristic that they are activated by the binding of IL-10 family cytokine, AK155, to the α subunit of the receptor; (2) bind to antibodies, e.g., polyclonal antibodies, raised against an immunogen comprising an amino acid sequence of SEQ ID NO:1 or SEQ ID NO. 2 and conservatively modified variants thereof; (3) specifically hybridize under stringent hybridization conditions to a sequence of SEQ ID NOS:7 and 8 and conservatively modified variants thereof; or (4) are amplified by primers that specifically hybridize under stringent hybridization conditions to the same sequence as a primer set selected from the group consisting of SEQ ID NOS:3-6.

“Cytokine” refers to small, biologically active, proteins produced by cells which act as intercellular mediators. Examples of cytokines include lymphokines, interleukins, interferons, and chemokines.

“IL-10-related cytokines” refers to cytokines with limited homology to IL-10. These cytokines include IL-20, IL-22, IL-19, melanoma differentiation-associated gene 7 (mda-7), and AK155 (Dumoutier, et al. (2001) J. Immunol. 167:3545-3549). The IL-10-related cytokines also include the IL-10 homologues of Epstein-Barr virus, equine herpesvirus type 2, and parapoxyirus (Knappe, et al. (2000) J. Virology 74:3881-3887)

“Recombinant” when used with reference, e.g., to a nucleic acid, plasmid, vector, virus, cell, or the like, indicates that these have been modified by the introduction of an exogenous, non-native nucleic acid or the alteration of a native nucleic acid, or have been derived from a recombinant nucleic acid, cell, virus, plasmid, or vector. “Recombinant protein” can refer to a protein derived from a recombinant nucleic acid, plasmid, vector, or cell, or the like, depending on the context.

“Exogenous” refers to substances that are produced outside an organism or cell, depending on the context.

“Inflammation” refers to a response to cellular injury that is marked by capillary dilation and leukocyte infiltration out of the capillaries and into the surrounding tissues. The classic signs of inflammation are: rubor (redness), tumor (swelling), calor (heat), and dolor (pain).

“Anti-inflammatory” refers to reducing inflammation by acting on body mechanisms without directly antagonizing the causative agent.

“Functional effects” in the context of assays for testing compounds affecting a receptor comprising the AK155 receptor includes the determination of any parameter that is indirectly or directly under the influence of the receptor. It includes physical and chemical effects, e.g., changes in ligand binding, and also other physiologic effects such as increases or decreases of transcription or hormone release.

“Inhibitors,” “activators” of the AK155 receptor refer to inhibitory or activating molecules, respectively, identified using in vitro and in vivo assays for AK155 receptor activation by AK155. A “modulator” of AK155 receptor activation is a molecule that is an inhibitor or an activator of AK155 receptor activation. Inhibitors are compounds that decrease, block, prevent, delay activation, inactivate, desensitize, or down regulate the receptor. Activators are compounds that increase, activate, facilitate, enhance activation, sensitize or up regulate receptor activity. Such assays for inhibitors and activators include e.g., expressing the AK155 receptor in cells and then measuring changes in the expression of certain molecules, including, without limitation, IL-8, IL-10, ICAM-1, ICAM-2 and B7-H1 (GenBank Accession No. XM016319). Alternatively, cells expressing endogenous AK155 receptor can be used in such assays. To examine the extent of inhibition, samples or assays comprising an AK155 receptor are treated with a potential activator or inhibitor and are compared to control samples without the inhibitor. Control samples (untreated with inhibitors) are assigned a relative AK155 receptor activity value of 100%. Inhibition of the AK155 receptor is achieved when the AK155 receptor activity value relative to the control is about 90%, preferably 50%, more preferably 25-0%. Activation of the AK155 receptor is achieved when the AK155 receptor activity value relative to the control is 110%, more preferably 150%, most preferably at least 200-500% higher or 1000% or higher.

“Biologically active” AK155 receptor refers to a heteromeric polypeptide comprised of one subunit having 75% or more amino acid identity to SEQ. ID No. 1 and a second subunit having 75% or more amino acid identity to SEQ ID No. 2 that has the ability to form a type II cytokine receptor having the characteristic that it binds AK155 as described above.

By “purified” and “isolated” is meant, when referring to a polypeptide, that the polypeptide is present in the substantial absence of the other biological macromolecules of the same type. The term “purified” as used herein means typically about 70%, more typically 75%, at least 80%, ordinarily 85%, more ordinarily 90%, preferably 95%, more preferably 98% by weight, or greater, of biological macromolecules of the same type present. The weights of water, buffers, salts, detergents, reductants, protease inhibitors, stabilizers, excipients, and other small molecules, especially those having a molecular weight of less than 1000, are not used in the determination of polypeptide purity (U.S. Pat. No. 6,090,611 issued to Covacci, et al.). Purity and homogeneity are typically determined using sodium dodecyl sulfate polyacrylamide gel electrophoresis (SDS PAGE), optionally with analysis using a gel scanner (Brody (1997) Analyt. Biochem. 247:247-256), high pressure liquid chromatography (HPLC) or capillary electrophoresis (CE) (Gooding and Regnier (2002) HPLC of Biological Molecules, 2^{nd} ed., Marcel Dekker, NY; Cunico, Gooding, and Wehr (1998) Basic HPLC and CE of Biomolecules, Bay Biological Laboratory, Inc. Hercules, Calif.), and N-terminal (Abe, et al. (1998) J. Biol. Chem. 273:11150-11157) or C-terminal amino acid sequencing (Boyd, et al. (1992) Analyt. Biochem. 206:344-352). A protein that is the predominant species present in a preparation is substantially purified. It is understood that 100% purity may be an impossibility, e.g., because of trace amounts of proteases or deamidation (Hsu, et al. (1998) Biochemistry 37:2251-2262; Robinson and Robinson (2001) Proc. Natl. Acad. Sci. USA 98:12409-12413; Sarioglu, et al. (2000) Electrophoresis 21:2209-2218). The influence of deamidation, limited proteolysis, and other forms of minor forms impurity, on antigenicity, biological activity, or therapeutic activity of any polypeptide or antibody is determined on an individual basis.

“Nucleic acid” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., (1991) Nucleic Acid Res. 19:5081; Ohtsuka, et al. (1985) J. Biol. Chem. 260:2605-2608; Rossolini et al. (1994) Mol. Cell. Probes 8:91-98). The term nucleic acid is used interchangeably with gene, cDNA, mRNA, oligonucleotide, and polynucleotide.

A particular nucleic acid sequence also implicitly encompasses “splice variants.”Similarly, a particular protein encoded by a nucleic acid implicitly encompasses any protein encoded by a splice variant of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

“Amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-TUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a conserved amino acid or a small percentage of amino acids in the encoded sequence is a “conservatively modified variant.” Conservative substitution tables providing functionally similar amino acids are well known in the art. An example of a conservative subtitution is the exchange of an amino acid in one of the following groups for another amino acid of the same group (U.S. Pat. No. 5,767,063 issued to Lee, et al.; Kyte and Doolittle (1982) J. Mol. Biol. 157:105-132).

(1) Hydrophobic: Norleucine, Ile, Val, Leu, Phe; Cys; or Met;

(2) Neutral hydrophilic: Cys, Ser, Thr;

(3) Acidic: Asp, Glu;

(4) Basic: Asn, Gln, H is, Lys, Arg;

(5) Residues that influence chain orientation: Gly; Pro;

(6) Aromatic: Trp, Tyr, Phe.

(7) Small amino acids: Gly, Ala, Ser.

For example, when no change or an insignificant change in biological function is desired, substitutions can be made by changing Val to Leu; Ser to Thr; or Asp to Glu, and the like.

Macromolecular structures such as polypeptide structures can be described in terms of various levels of organization. For a general discussion of this organization, see, e.g., Alberts et al., Molecular Biology of the Cell (3^{rd} ed., 1994) and Cantor and Schimmel, Biophysical Chemistry Part I: The Conformation of Biological Macromolecules (1980). “Primary structure” refers to the amino acid sequence of a particular peptide. “Secondary structure” refers to locally ordered, three dimensional structures within a polypeptide. These structures are commonly known as domains. Domains are portions of a polypeptide that form a compact unit of the polypeptide and are typically 50 to 350 amino acids long. Typical domains are made up of sections of lesser organization such as stretches of β-sheet and α-helices. “Tertiary structure” refers to the complete three dimensional structure of a polypeptide monomer. “Quaternary structure” refers to the three dimensional structure formed by the noncovalent association of independent tertiary units. Anisotropic terms are also known as energy terms.

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription.

A “constitutive” promoter is a promoter that is active under most environmental and developmental conditions.

An “inducible” promoter is a promoter that is active under environmental or developmental regulation.

The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

An “expression vector” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed operably linked to a promoter.

The terms “identical” or percent “identity,” in the context of two or more nucleic acids or polypeptide sequences, refer to two or more sequences or subsequences that are the same or have a specified percentage of amino acid residues or nucleotides that are the same i.e., 65% identity, preferably 70%, 75%, 80%, 85%, 90%, or 95% identity over a specified region when compared and aligned for maximum correspondence over a comparison window, or designated region as measured using one of the following sequence comparison algorithms or by manual alignment and visual inspection. Such sequences are then said to be “substantially identical.” This definition also refers to the compliment of a test sequence. Preferably, the identity exists over a region that is at least about 25 amino acids or nucleotides in length, or more preferably over a region that is 50-100 amino acids or nucleotides in length. and most preferably the sequences are substantially identical over at least about 150 residues. In a most preferred embodiment, the sequences are substantially identical over the entire length of the coding regions and/or untranslated regions.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins to AK155 receptor nucleic acids and proteins, the BLAST and BLAST 2.0 algorithms and the default parameters discussed below are used.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman (1981) Adv. Appl. Math. 2:482, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

A preferred example of algorithm that is suitable for determining percent sequence identity and sequence similarity are the BLAST and BLAST 2.0 algorithms, which are described in Altschul et al. (1977) Nuc. Acids Res. 25:3389-3402 and Altschul et al. (1990) J. Mol. Biol. 215:403-410, respectively. BLAST and BLAST 2.0 are used, with the parameters described herein, to determine percent sequence identity for the nucleic acids and proteins of the invention. Software for performing BLAST analyses is publicly available through the National Center for Biotechnology Information, on the world wide web at “ncbi.nlm.nih.gov.” This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Nat'l. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For high stringency hybridization, a positive signal is at least two times background, preferably 10 times background hybridization. Exemplary high stringency or stringent hybridization conditions include: 50% formamide, 5× SSC and 1% SDS incubated at 42° C. or 5× SSC and 1% SDS incubated at 65° C., with a wash in 0.2× SSC and 0.1% SDS at 65° C.

Nucleic acids that do not hybridize to each other under stringent conditions are still substantially identical if the polypeptides that they encode are substantially identical. This occurs, for example, when a copy of a nucleic acid is created using the maximum codon degeneracy permitted by the genetic code. In such cased, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1× SSC at 45° C. A positive hybridization is at least twice background. Those of ordinary skill will readily recognize that alternative hybridization and wash conditions can be utilized to provide conditions of similar stringency.

“Antibody” refers to a polypeptide comprising a framework region from an immunoglobulin gene or fragments thereof that specifically binds and recognizes an antigen. The immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region.

“Fv” is has been considered the minimum antibody fragment which contains a complete antigen-recognition and binding site. This region comprises a dimer of one heavy chain and one light chain variable domain in tight association with each other. It is in this configuration that the three CDRs of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

“Diabodies” refers to small antibody fragments with two antigen-binding sites, which fragments comprise a heavy chain variable domain (V_{H}) connected to a light chain variable domain (V_{L}) on the same polypeptide chain (V_{H}-V_{L}) (Holliger, et al. (1993) Proc. Natl. Acad. Sci. USA 90:6444-6448).

An “immunoassay” is an assay that uses an antibody or antibody fragment to specifically bind an antigen. The immunoassay is characterized by the use of specific binding properties of a particular antibody to isolate, target, or quantify the antigen.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

“Soluble receptor” refers to receptors that are water-soluble and occur, e.g., in extracellular fluids, intracellular fluids, or weakly associated with a membrane. Soluble receptor also refers to receptors that are released from tight association with a membrane, e.g., by limited cleavage. Soluble receptor further refers to receptors that are engineered to be water soluble. See, e.g., Monahan, et al. (1997) J. Immunol. 159:4024-4034; Moreland, et al. (1997) New Engl. J. Med. 337:141-147; Borish, et al. (1999) Am. J. Respir. Crit. Care Med. 160:1816-1823; Uchibayashi, et al. (1989) J. Immunol. 142:3901-3908.

The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. For example, polyclonal antibodies raised to the AK155 alpha subunit, as shown in SEQ ID NO. 1, or splice variants, or portions thereof, can be selected to obtain only those polyclonal antibodies that are specifically immunoreactive with the AK155 receptor and not with other proteins, except for polymorphic variants, orthologs, and alleles of the AK155 receptor. A variety of immunoassay formats may be used to select antibodies specifically immunoreactive with a particular protein. For example, solid-phase ELISA immunoassays are routinely used to select antibodies specifically immunoreactive with a protein (see, e.g., Harlow and Lane, Antibodies, A Laboratory Manual (1988) for a description of immunoassay formats and conditions that can be used to determine specific immunoreactivity). Typically a specific or selective reaction will be at least twice background signal or noise and more typically more than 10 to 100 times background.

By “host cell” is meant a cell that contains an expression vector and supports the replication or expression of the expression vector. Host cells may be prokaryotic cells such as E. coli, or eukaryotic cells such as yeast, insect, amphibian, or mammalian cells such as Ba/F3, COLO205 and the like, e.g., cultured cells, explants, and cells in vivo.

“Biological sample” as used herein is a sample of biological tissue or fluid that contains the AK155 receptor or nucleic acid encoding the subunits of the AK155 receptor proteins. Such samples include, but are not limited to, tissue isolated from humans. Biological samples may also include sections of tissues such as frozen sections taken for histologic purposes. A biological sample is typically obtained from a eukaryotic organism, preferably eukaryotes such as fungi, plants, insects, protozoa, birds, fish, reptiles, and preferably a mammal such as rat, mice, cow, dog, guinea pig, or rabbit, and most preferably a primate such as chimpanzees or humans.

The phrase “specifically” or “selectively” binds, when referring to a ligand/receptor or other binding pair, refers to a binding reaction which is determinative of the presence of the protein in the presence of a heterogeneous population of proteins and other biologics. Thus, under designated conditions, a specified ligand binds to a particular receptor and does not bind in a significant amount to other proteins present in the sample. Preferred binding agents bind with an affinity of at least about 1 μM, more preferably of at least about 100 nM, and still more preferably of at least about 10 nM, and even more preferably of at least about 1 nM.

“Ligand” refers to an entity that specifically binds to a polypeptide or a complex of more than one polypeptide. A “ligand binding domain” is a polypeptide or region of a polypeptide that is able to bind to said entity. A ligand may be a soluble protein, a membrane-associated protein, or an integral membrane-bound protein. Where a ligand binds to a receptor, the question of which molecule is the ligand and which molecule is the receptor may be determined on a case-by-case basis. Generally, where the binding event results in cell signaling, any molecule that is constitutively bound to the signaling cell is considered to be part of the receptor, and not part of the ligand. A freely diffusable and water-soluble entity that is involved in ligand/receptor interactions is usually a ligand, not a receptor.

“Activity” of a molecule may describe or refer to binding of the molecule to a ligand or to a receptor, to catalytic activity, to the ability to stimulate gene expression, to antigenic activity, to the modulation of activities of other molecules, and the like. “Activity” may also mean specific activity, e.g., catalytic activity/total protein in a sample, or immunological activity/total protein in a sample, or the like.

An “agonist” is a compound that interacts with a target or that can cause an increase in the activation of the target.

An “antagonist” is a compound that opposes the actions of an agonist

As used herein a “nucleic acid probe or oligonucleotide” is defined as a nucleic acid capable of binding to a target nucleic acid of complementary sequence through one or more types of chemical bonds, usually through complementary base pairing, usually through hydrogen bond formation. As used herein, a probe may include natural (i.e., A, G, C, or T) or modified bases (7-deazaguanosine, inosine, etc.). In addition, the bases in a probe may be joined by a linkage other than a phosphodiester bond, so long as it does not interfere with hybridization. Thus, for example, probes may be peptide nucleic acids in which the constituent bases are joined by peptide bonds rather than phosphodiester linkages. It will be understood by one of skill in the art that probes may bind target sequences lacking complete complementarity with the probe sequence depending upon the stringency of the hybridization conditions. By assaying for the presence or absence of the probe, one can detect the presence or absence of the select sequence or subsequence.

A “labeled nucleic acid probe or oligonucleotide” is one that is bound, either covalently, through a linker or a chemical bond, or noncovalently, through ionic, van der Waals, electrostatic, or hydrogen bonds to a label such that the presence of the probe may be detected by detecting the presence of the label bound to the probe. The probes are preferably directly labeled as with isotopes, chromophores, fluorophores, chromogens, or indirectly labeled such as with biotin to which a streptavidin complex may later bind.

A composition is “labeled” that is detectable, either directly or indirectly, by spectroscopic, photochemical, biochemical, immunochemical, or chemical means. For example, useful labels include ³²P, fluorescent dyes, electron-dense reagents, enzymes and their substrates (e.g., as commonly used in enzyme-linked immunoassays, e.g., alkaline phosphatase and horse radish peroxidase), biotin-streptavidin, digoxigenin, or haptens and proteins for which antisera or monoclonal antibodies are available. The label or detectable moiety is typically bound, either covalently, through a linker or chemical bound, or through ionic, van der Waals or hydrogen bonds to the molecule to be detected.

The term “radiolabeled” refers to a compound to which a radioisotope has been attached through covalent or non-covalent means. Examples of radioisotopes include, without limitation, H³, P³³, P³², S35, and I¹²⁵.

A “fluorophore” is a compound or moiety that accepts radiant energy of one wavelength and emits radiant energy of a second wavelength.

### DETAILED DESCRIPTION

As used herein, including the appended claims, the singular forms of words such as “a,” “an,” and “the” include their corresponding plural references unless the context clearly dictates otherwise.

Unless otherwise defined, all technical and scientific terms used herein have the same meaning as commonly understood by a person of ordinary skill in the art to which this invention belongs. Although methods and materials similar or equivalent to those described herein can be used in the practice or testing of the present invention, suitable methods and materials are described below. All publications, patent applications, patents, and other references discussed herein are provided solely for their disclosure prior to the filing date of the present application. Nothing herein is to be construed as an admission that the invention is not entitled to antedate any such disclosure by virtue of its prior invention. All publications, patent applications, patents, and other references mentioned herein are incorporated by reference in their entirety including all figures, graphs, and drawings.

All citations herein are incorporated herein by reference to the same extent as if each individual publication or patent application was specifically and individually indicated to be incorporated by reference.

I. Introduction.

The present invention is based on the identification of the interleukin AK155, (see e.g., U.S. Pat. No. 5,989,867; Knappe et al. (2000) J. Virol. 74:3881-3887) as a ligand for the AK155 receptor. Thus, the present invention provides for cells expressing the AK155 receptor i.e., SEQ ID NO: 1 (GenBank Accession No. AF184971, also called IL-20Rα; IL-20R1) and SEQ ID NO: 2 (GenBank Accession No. NM_{—}000628, also called IL-10Rβ; IL-10R2) in combination as a complex. In addition, the present invention provides for methods for identifying therapeutic agents (e.g., anti-inflammatory agents, anti-angiogenesis agents, anti-cancer agents, etc.) that modulate AK155's effects on an AK155 receptor, such as modulators, agonists, antagonists, etc. Such therapeutic agents are useful for treating AK155 mediated-conditions or diseases, such as inflammation, angiogenesis, and cancer.

Thus, the invention also provides a method of inhibiting inflammation in a patient suffering from inflammatory disease, the method comprising the administration of an antagonist of the AK155-AK155-receptor complex in an amount effective to inhibit AK155 activated inflammation. The methods and components of the present invention will be described in more detail below.

II. Methods for Identifying Compounds for use in Treating Ak155—Mediated Disorders and Conditions.

The invention provides methods for identifying compounds that can modulate AK155—and/or AK155 receptor-mediated processes (e.g., inflammation, angiogenesis, and cell growth). These methods involve testing candidate therapeutic agents to determine whether the candidate therapeutic agent can modulate an AK155—mediated process. If the candidate therapeutic agent modulates an AK155 process, then it can be used as a therapeutic agent to treat an AK155—and/or AK155 receptor-mediated disease or condition, such as cancer, inflammatory diseases of the gut (Crohn's disease; colitis; coeliac disease), autoimmune diseases (multiple sclerosis; diabetes mellitus; Sjogren's syndrome); inflammatory diseases of the skin (psoriasis; lupus erythematosus; vitiligo; atopic eczema; atopic dermatitis), IgE-dependent diseases (asthma; anaphylaxis; allergic rhinitis), immune-related diseases of muscle (myasthenia gravis); transplant-related immune diseases (transplant rejection; graft versus host disease), immune-related diseases of the joints (rheumatoid arthritis), and autoimmune diseases of the connective tissue (Sjogren's syndrome; scleroderma; polymyositis, and systemic lupus erythematosus).

In certain embodiments, the candidate therapeutic agents are pre-screened in one or more in vitro assays to identify those compounds or agents can modulate the interaction between an AK155 and an AK155 receptor, or modulate the effects of AK155 on a physiological process, etc. The potential candidate therapeutic agents can be screened in vitro before in vivo testing. If the candidate therapeutic agent is active in an in vitro screening assay, then the candidate therapeutic agent is more likely to affect an AK155—and/or AK155 receptor-mediated process in vivo. The methods and components of these screening assays will be described in more detail below.

A. AK155 Polypeptides.

AK155 polypeptides for use in the screening assays can be prepared using methods that are known in the art (see e.g., U.S. Pat. No. 5,989,867; Knappe et al. (2000) J. Virol. 74:3881-3887). Nucleic acids that encode AK155 have been described in, for example, U.S. Pat. No. 5,989,867; Knappe et al. (2000) J. Virol. 74:3881-3887; GenBank Accession No. AJ251549, and SEQ ID NO: 10).

Polynucleotide vectors that facilitate the expression of fusion proteins are commercially available (e.g., New England Biolabs, Invitrogen and Novagen). For example, a histidine tagged AK155 can be expressed in E. coli and purified over an immobilized metal affinity column (see e.g., Current Protocols in Molecular Biology (Ausubel et al., eds, 1994). Other fusion partners are well known in the art and can be used to express an AK155 fusion protein.

B. AK155 Receptors.

Nucleic acids encoding an AK155 receptor (AK155R) can be isolated using the methods described below.

1. General Recombinant DNA Methods.

This invention relies on routine techniques in the field of recombinant genetics. Basic texts disclosing the general methods of use in this invention include Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994)).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kD) or amino acid residue numbers. Proteins sizes are estimated from gel electrophoresis, from sequenced proteins, from derived amino acid sequences, or from published protein sequences.

Oligonucleotides that are not commercially available can be chemically synthesized according to the solid phase phosphoramidite triester method first described by Beaucage and Caruthers (1981) Tetrahedron Letts. 22:1859-1862, using an automated synthesizer, as described in Van Devanter et. al. (1984) Nucleic Acids Res. 12:6159-6168. Purification of oligonucleotides is by either native acrylamide gel electrophoresis or by anion-exchange HPLC as described in Pearson and Reanier (1983) J. Chrom. 255:137-149.

The sequence of the cloned genes and synthetic oligonucleotides can be verified after cloning using, e.g., the chain termination method for sequencing double-stranded templates of Wallace et al. (1981) Gene 16:21-26.

2. Cloning Methods for the Isolation of Nucleotide Sequences Encoding α- and β-subunits of the AK155 Receptor.

In general, the nucleic acid sequences encoding AK155 receptor and related nucleic acid sequence homologs are cloned from cDNA and genomic DNA libraries or isolated using amplification techniques with oligonucleotide primers. For example, AK155 receptor sequences are typically isolated from human nucleic acid (genomic or cDNA) libraries by hybridizing with a nucleic acid probe or polynucleotide, the sequence of which can be derived from SEQ ID NOS:7-8, preferably from a conserved region. Suitable source from which AK155 receptor RNA and cDNA can be isolated include, but are not limited to, Colo-205 (colon carcinoma), SW-403 (colon carcinoma), Lovo (colon carcinoma), and HaCaT (keratinocytes, IL-20 reactive) cells.

Amplification techniques using primers can also be used to amplify and isolate AK155 receptor subunits from DNA or RNA. The following primers can also be used to amplify a sequence of the α subunit of the hAK155 receptor: 5′-ATGAAGAATGTCCTACAATGGACTCC (SEQ ID No. 3) and 5′-TCAGTTTTCCATCTGCACATATAACC (SEQ ID No. 4). The β subunit can be amplified with the following primers: 5′-ATGGCGTGGAGTCTTGGGA (SEQ ID No. 5) and 5′-TCACTCCATCCTACCTACCTCTTTCA (SEQ ID No. 6).

These primers can be used, e.g., to amplify the full length sequence or a probe, which is then used to screen a library for full-length AK155 receptor.

Nucleic acids encoding AK155 receptor subunits can also be isolated from expression libraries using antibodies as probes. Such polyclonal or monoclonal antibodies can be raised using the sequence of SEQ ID NO:1 or SEQ ID NO:2, or an immunogenic portion thereof.

Polymorphic variants of the AK155 receptor subunits, orthologs, and alleles that are substantially identical to the conserved regions of AK155 receptor subunits (e.g., SEQ ID NOS: 11-16) can be isolated using AK155 receptor nucleic acid probes and oligonucleotides under stringent hybridization conditions, by screening libraries. Alternatively, expression libraries can be used to clone AK155 receptor subunits and receptor polymorphic variants, orthologs, and alleles by detecting expressed homologs immunologically with antisera or purified antibodies made against AK155 receptor subunits or portions thereof (e.g., the conserved regions of AK155 receptor subunits), which also recognize and selectively bind to the AK155 receptor subunit homologs.

To make a cDNA library, one should choose a source that is rich in AK155 receptor mRNA, e.g., Colo-205 (colon carcinoma), SW-403 (colon carcinoma), Lovo (colon carcinoma), and HaCaT (keratinocytes, IL-20 reactive) cells. The mRNA is then made into cDNA using reverse transcriptase, ligated into a recombinant vector, and transfected into a recombinant host for propagation, screening and cloning. Methods for making and screening cDNA libraries are well known (see, e.g., Gubler and Hoffman (1983) Gene 25:263-269; Sambrook et al., supra; Ausubel et al., supra).

For a genomic library, the DNA is extracted from the tissue and either mechanically sheared or enzymatically digested to yield fragments of about 12-20,kb. The fragments are then separated by gradient centrifugation from undesired sizes and are constructed in bacteriophage lambda vectors. These vectors and phage are packaged in vitro. Recombinant phage are analyzed by plaque hybridization as described in Benton and Davis (1977) Science 196:180-182. Colony hybridization is carried out as generally described in Grunstein et al. (1975) Proc. Natl. Acad. Sci. USA., 72:3961-3965.

An alternative method of isolating AK155 receptor nucleic acids and its orthologs, alleles, mutants, polymorphic variants, and conservatively modified variants combines the use of synthetic oligonucleotide primers and amplification of an RNA or DNA template (see U.S. Pat. Nos. 4,683,195 and 4,683,202; PCR Protocols: A Guide to Methods and Applications (Innis et al., eds, 1990)). Methods such as polymerase chain reaction (PCR) and ligase chain reaction (LCR) can be used to amplify nucleic acid sequences of AK155 receptor directly from mRNA, from cDNA, from genomic libraries or cDNA libraries. Degenerate oligonucleotides can be designed to amplify AK155 receptor homologs using the sequences provided herein. Restriction endonuclease sites can be incorporated into the primers. Polymerase chain reaction or other in vitro amplification methods may also be useful, for example, to clone nucleic acid sequences that code for proteins to be expressed, to make nucleic acids to use as probes for detecting the presence of AK 155 receptor encoding mRNA in physiological samples, for nucleic acid sequencing, or for other purposes. Genes amplified by the PCR reaction can be purified from agarose gels and cloned into an appropriate vector.

Gene expression of AK155 receptor can also be analyzed by techniques known in the art, e.g., reverse transcription and amplification of mRNA, isolation of total RNA or poly A⁺ RNA, northern blotting, dot blotting, in situ hybridization, RNase protection, high density polynucleotide array technology and the like.

Synthetic oligonucleotides can be used to construct recombinant AK155 receptor genes for use as probes or for expression of protein. This method is performed using a series of overlapping oligonucleotides usually 40-120 bp in length, representing both the sense and nonsense strands of the gene. These DNA fragments are then annealed, ligated and cloned. Alternatively, amplification techniques can be used with precise primers to amplify a specific subsequence of the AK155 receptor genes. The specific subsequence is then ligated into an expression vector.

The genes for the α and β chains of the AK155 receptor are typically cloned into intermediate vectors before transformation into prokaryotic or eukaryotic cells for replication and/or expression. These intermediate vectors are typically prokaryote vectors, e.g., plasmids, or shuttle vectors.

3. Expression in Prokaryotes and Eukaryotes.

To obtain high level expression of a cloned gene, such as those cDNAs encoding the AK155 receptor, one typically subclones the AK155 receptor subunits into an expression vectors that contain a strong promoter to direct transcription, a transcription/translation terminator, and if for a nucleic acid encoding a protein, a ribosome binding site for translational initiation. Suitable bacterial promoters are well known in the art and described, e.g., in Sambrook et al., and Ausubel et al, supra. Bacterial expression systems for expressing the AK155 receptor protein are available in, e.g., E. coli, Bacillus sp., and Salmonella (Palva et al. (1983) Gene 22:229-235; Mosbach et al. (1983) Nature 302:543-545. Kits for such expression systems are commercially available. Eukaryotic expression systems for mammalian cells, yeast, and insect cells are well known in the art and are also commercially available.

Selection of the promoter used to direct expression of a heterologous nucleic acid depends on the particular application. The promoter is preferably positioned about the same distance from the heterologous transcription start site as it is from the transcription start site in its natural setting. As is known in the art, however, some variation in this distance can be accommodated without loss of promoter function.

In addition to the promoter, the expression vector typically contains a transcription unit or expression cassette that contains all the additional elements required for the expression of the AK155 receptor subunit encoding nucleic acid in host cells. A typical expression cassette thus contains a promoter operably linked to the nucleic acid sequence encoding AK155 receptor subunit and signals required for efficient polyadenylation of the transcript, ribosome binding sites, and translation termination. Additional elements of the cassette may include enhancers and, if genomic DNA is used as the structural gene, introns with functional splice donor and acceptor sites.

In addition to a promoter sequence, the expression cassette should also contain a transcription termination region downstream of the structural gene to provide for efficient termination. The termination region may be obtained from the same gene as the promoter sequence or may be obtained from different genes.

The particular expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, pET23D, and fusion expression systems such as MBP, GST, and LacZ. Epitope tags can also be added to recombinant proteins to provide convenient methods of isolation, e.g., c-myc.

Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A⁺, pMTO10/A⁺, pMAMneo-5, baculovirus pDSVE, and any other vector allowing expression of proteins under the direction of the CMV promoter, SV40 early promoter, SV40 later promoter, metallothionein promoter, murine mammary tumor virus promoter, Rous sarcoma virus promoter, polyhedrin promoter, or other promoters shown effective for expression in eukaryotic cells.

Some expression systems have markers that provide gene amplification such as thymidine kinase and dihydrofolate reductase. Alternatively, high yield expression systems not involving gene amplification are also suitable, such as using a baculovirus vector in insect cells, with a AK155 receptor encoding sequence under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are preferably chosen such that they do not interfere with the replication of the DNA in eukaryotic cells, if necessary.

Standard transfection methods are used to produce bacterial, mammalian, yeast or insect cell lines that express large quantities of AK155 receptor proteins, which are then purified using standard techniques (see, e.g., Colley et al. (1989) J. Biol. Chem. 264:17619-17622; Guide to Protein Purification, in Methods in Enzymology, vol. 182 (Deutscher, ed., 1990)). Transformation of eukaryotic and prokaryotic cells are performed according to standard techniques (see, e.g., Morrison (1977) J. Bact. 132:349-351; Clark-Curtiss and Curtiss (1983) Methods in Enzymology 101:347-362 (Wu et al., eds,).

Any of the well-known procedures for introducing foreign nucleotide sequences into host cells may be used. These include the use of calcium phosphate transfection, polybrene, protoplast fusion, electroporation, biolistics, liposomes, microinjection, plasma vectors, viral vectors and any of the other well known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least both genes into the host cell capable of expressing the AK155 receptor.

After the expression vector is introduced into the cells, the transfected cells are cultured under conditions favoring expression of the AK155 receptor subunits, which is recovered from the culture using standard techniques identified below.

4. Immunological Detection of AK155 Receptor and the Products of its Activation.

In addition to the detection of the AK155 receptor genes and gene expression using nucleic acid hybridization technology, one can also use immunoassays to detect the AK155 receptor subunits or the peptide products that result from activation of the AK155 receptor. Immunoassays can be used to qualitatively or quantitatively analyze the AK155 receptor subunits and other peptides. A general overview of the applicable technology can be found in Harlow and Lane, Antibodies: A Laboratory Manual (1988).

Methods of producing polyclonal and monoclonal antibodies that react specifically with the AK155 receptor subunits or the protein products that result from AK155 receptor activation, are known to those of skill in the art (see, e.g., Coligan, Current Protocols in Immunology (1991); Harlow and Lane, supra; Goding, Monoclonal Antibodies: Principles and Practice (2d ed. 1986); and Kohler and Milstein (1975) Nature 256:495-497. Such techniques include antibody preparation by selection of antibodies from libraries of recombinant antibodies in phage or similar vectors, as well as preparation of polyclonal and monoclonal antibodies by immunizing rabbits or mice (see, e.g., Huse et al. (1989) Science 246:1275-1281; Ward et al. (1989) Nature 341:544-546).

A number of immunogens comprising portions of proteins may be used to produce antibodies specifically reactive with the protein of interest. For example, recombinant proteins antigenic fragments thereof, can be isolated as described herein. Recombinant proteins can be expressed in eukaryotic or prokaryotic cells as described above, and purified as generally described above. Recombinant protein is the preferred immunogen for the production of monoclonal or polyclonal antibodies. Alternatively, a synthetic peptide derived from the sequences disclosed herein and conjugated to a carrier protein can be used an immunogen. Naturally occurring protein may also be used either in pure or impure form. The product is then injected into an animal capable of producing antibodies. Either monoclonal or polyclonal antibodies may be generated, for subsequent use in immunoassays to measure the protein.

Methods of production of polyclonal antibodies are known to those of skill in the art. An inbred strain of mice (e.g., BALB/C mice) or rabbits is immunized with the protein using a standard adjuvant, such as Freund's adjuvant, and a standard immunization protocol. The animal's immune response to the immunogen preparation is monitored by taking test bleeds and determining the titer of reactivity to the beta subunits. When appropriately high titers of antibody to the immunogen are obtained, blood is collected from the animal and antisera are prepared. Further fractionation of the antisera to enrich for antibodies reactive to the protein can be done if desired (see, Harlow and Lane, supra).

Monoclonal antibodies may be obtained by various techniques familiar to those skilled in the art. Briefly, spleen cells from an animal immunized with a desired antigen are immortalized, commonly by fusion with a myeloma cell (see, Kohler and Milstein (1976) Eur. J. Immunol. 6:511-519). Alternative methods of immortalization include transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according to the general protocol outlined by Huse, et al. (1989) Science 246:1275-1289.

Monoclonal antibodies and polyclonal sera are collected and titered against the immunogen protein in an immunoassay, for example, a solid phase immunoassay with the immunogen immobilized on a solid support. Typically, polyclonal antisera with a titer of 104 or greater are selected and tested for their cross reactivity against related proteins using a competitive binding immunoassay. Specific polyclonal antisera and monoclonal antibodies will usually bind with a K_{d} of at least about 0.1 mM, more usually at least about 1 mM, preferably at least about 0.1 mM or better, and most preferably, 0.01 mM or better.

Once the specific antibodies against a protein are available, the protein can be detected by a variety of immunoassay methods. For a review of immunological and immunoassay procedures, see Basic and Clinical Immunology (Stites and Terr eds., 7^{th} ed. 1991). Moreover, the immunoassays of the present invention can be performed in any of several configurations, which are reviewed extensively in Enzyme Immunoassay (Maggio, ed., 1980); and Harlow and Lane, supra.

Proteins can be detected and/or quantified using any of a number of well recognized immunological binding assays (see, e.g., U.S. Pat. Nos. 4,366,241; 4,376,110; 4,517,288; and 4,837,168). For a review of the general immunoassays, see also Methods in Cell Biology: Antibodies in Cell Biology, volume 37 (Asai, ed. 1993); Basic and Clinical Immunology (Stites and Terr, eds., 7^{th} ed. 1991). Immunological binding assays (or immunoassays) typically use an antibody that specifically binds to a protein or antigen of choice (in this case the AK155 receptor or the products of its activation). The antibody may be produced by any of a number of means well known to those of skill in the art and as described above.

Immunoassays also often use a labeling agent to specifically bind to and label the complex formed by the antibody and antigen. The labeling agent may itself be one of the moieties comprising the antibody/antigen complex. Thus, the labeling agent may be a labeled antigen or a labeled antibody. Alternatively, the labeling agent may be a third moiety, such a secondary antibody, which specifically binds to the antibody/antigen complex (a secondary antibody is typically specific to antibodies of the species from which the first antibody is derived). Other proteins capable of specifically binding immunoglobulin constant regions, such as protein A or protein G may also be used as the label agent. These proteins exhibit a strong non-immunogenic reactivity with immunoglobulin constant regions from a variety of species (see, e.g., Kronval et al. (1973) J. Immunol. 111: 1401-1406; Akerstrom et al. (1985) J. Immunol. 135:2589-2542). The labeling agent can be modified with a detectable moiety, such as biotin, to which another molecule can specifically bind, such as streptavidin. A variety of detectable moieties are well known to those skilled in the art.

Throughout the assays, incubation and/or washing steps may be required after each combination of reagents. Incubation steps can vary from about 5 seconds to several hours, preferably from about 5 minutes to about 24 hours. However, the incubation time will depend upon the assay format, antigen, volume of solution, concentrations, and the like. Usually, the assays will be carried out at ambient temperature, although they can be conducted over a range of temperatures, such as 10° C. to 40° C.

Immunoassays for detecting the AK155 receptor subunits and the products of AK155 receptor activation in samples may be either competitive or noncompetitive. Noncompetitive immunoassays are assays in which the amount of antigen is directly measured. In one preferred “sandwich” assay, for example, AK155 receptor antibodies can be bound directly to a solid substrate on which they are immobilized. These immobilized antibodies then capture an AK155 receptor (or an AK155 receptor subunit) present in the test sample. The AK155 receptor is thus immobilized and then bound by a labeling agent, such as a second AK155 receptor antibody bearing a label. Alternatively, the second antibody may lack a label, but it may, in turn, be bound by a labeled third antibody specific to antibodies of the species from which the second antibody is derived. The second or third antibody is typically modified with a detectable moiety, such as biotin, to which another molecule specifically binds, e.g., streptavidin, to provide a detectable moiety.

i. Competitive Assay Formats.

In competitive assays, the amount of AK155 receptor present in the sample is measured indirectly by measuring the amount of known, added (exogenous) AK155 receptor displaced (competed away) from an anti- AK155 receptor antibody by the unknown AK155 receptor present in a sample. In one competitive assay, a known amount of AK155 receptor is added to a sample and the sample is then contacted with an antibody that specifically binds to AK155 receptor. The amount of exogenous AK155 receptor bound to the antibody is inversely proportional to the concentration of the AK155 receptor present in the sample. In a particularly preferred embodiment, the antibody is immobilized on a solid substrate. The amount of AK155 receptor bound to the antibody may be determined either by measuring the amount of AK155 receptor present in a AK155 receptor/antibody complex, or alternatively by measuring the amount of remaining uncomplexed protein. The amount of AK155 receptor may be detected by providing a labeled AK155 receptor molecule.

A hapten inhibition assay is another preferred competitive assay. In this assay the known AK155 receptor is immobilized on a solid substrate. A known amount of anti-AK155 receptor antibody is added to the sample, and the sample is then contacted with the immobilized AK155 receptor. The amount of anti-AK155 receptor antibody bound to the known immobilized AK155 receptor is inversely proportional to the amount of AK155 receptor present in the sample. Again, the amount of immobilized antibody may be detected by detecting either the immobilized fraction of antibody or the fraction of the antibody that remains in solution. Detection may be direct where the antibody is labeled or indirect by the subsequent addition of a labeled moiety that specifically binds to the antibody as described above.

ii. Other Assay Formats.

Western blot (immunoblot) analysis is used to detect and quantify the presence of the AK155 receptor (or an AK155 receptor subunit), or the products of its activation (e.g., IL-8, IL-10, ICAM-1, ICAM-2 and B7-H1), in the sample. The technique generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with the antibodies that specifically bind the protein of interest. The antibodies specifically bind to the protein of interest on the solid support. These antibodies may be directly labeled or alternatively may be subsequently detected using labeled antibodies (e.g., labeled sheep anti-mouse antibodies) that specifically bind to the antibodies against the protein of interest.

Other assay formats include liposome immunoassays (LIA), which use liposomes designed to bind specific molecules (e.g., antibodies) and release encapsulated reagents or markers. The released chemicals are then detected according to standard techniques (see, Monroe et al. (1986) Amer. Clin. Prod. Rev. 5:34-41).

iii. Reduction of Non-Specific Binding.

One of skill in the art will appreciate that it is often desirable to minimize non-specific binding in immunoassays. Particularly, where the assay involves an antigen or antibody immobilized on a solid substrate it is desirable to minimize the amount of non-specific binding to the substrate. Means of reducing such non-specific binding are well known to those of skill in the art. Typically, this technique involves coating the substrate with a proteinaceous composition. In particular, protein compositions such as bovine serum albumin (BSA), nonfat powdered milk, and gelatin are widely used with powdered milk being most preferred.

iv. Labels.

The particular label or detectable group used in the assay is not a critical aspect of the invention, as long as it does not significantly interfere with the specific binding of the antibody used in the assay. The detectable group can be any material having a detectable physical or chemical property. Such detectable labels have been well-developed in the field of immunoassays and, in general, most any label useful in such methods can be applied to the present invention. Thus, a label is any composition detectable by spectroscopic, photochemical, biochemical, immunochemical, electrical, optical or chemical means. Useful labels in the present invention include magnetic beads (e.g., Dynabeads®), fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold or colored glass or plastic beads (e.g., polystyrene, polypropylene, latex, etc.).

The label may be coupled directly or indirectly to the desired component of the assay according to methods well known in the art. As indicated above, a wide variety of labels may be used, with the choice of label depending on sensitivity required, ease of conjugation with the compound, stability requirements, available instrumentation, and disposal provisions.

Non-radioactive labels are often attached by indirect means. Generally, a ligand molecule (e.g., biotin) is covalently bound to the molecule. The ligand then binds to another molecule (e.g., streptavidin), which is either inherently detectable or covalently bound to a signal system, such as a detectable enzyme, a fluorescent compound, or a chemiluminescent compound. The ligands and their targets can be used in any suitable combination with antibodies that recognize the AK155 receptor, or the products of its activation, or secondary antibodies that recognize antibodies directed against the AK155 receptor, or the products of its activation.

The molecules can also be conjugated directly to signal generating compounds, e.g., by conjugation with an enzyme or fluorophore. Enzymes of interest as labels will primarily be hydrolases, particularly phosphatases, esterases and glycosidases, or oxidases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazinediones, e.g., luminol. For a review of various labeling or signal producing systems that may be used, see, U.S. Pat. No. 4,391,904.

Means of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence. The fluorescence may be detected visually, by means of photographic film, by the use of electronic detectors such as charge coupled devices (CCDs) or photomultipliers and the like. Similarly, enzymatic labels may be detected by providing the appropriate substrates for the enzyme and detecting the resulting reaction product. Finally, simple colorimetric labels may be detected simply by observing the color associated with the label. Thus, in various dipstick assays, conjugated gold often appears pink, while various conjugated beads appear the color of the bead.

Some assay formats do not require the use of labeled components. For instance, agglutination assays can be used to detect the presence of the target antibodies. In this case, antigen-coated particles are agglutinated by samples comprising the target antibodies. In this format, none of the components need be labeled and the presence of the target antibody is detected by simple visual inspection.

C. Cells that Express AK155 Receptors.

Cells that recombinantly express an AK155 receptor can be generated using standard molecular biology techniques to transfect expression cassettes encoding AK155 receptor subunits into an appropriate cell. Alternatively, cells that express an AK155 receptor can be identified by assaying for the effects of AK155 on the cell, using RT-PCR and other nucleic acid amplification techniques to detect AK155 receptor subunit mRNAs etc. Examples of cells that express an AK155 receptor include, without limitation, Colo-205 (colon carcinoma), SW-403 (colon carcinoma), Lovo (colon carcinoma), HaCaT (keratinocytes, IL-20 reactive) cells and the like.

D. Direct and Displacement Assays.

Candidate therapeutic agents can be screened for their ability to interact directly with a polypeptide that includes a AK155 ligand binding domain (e.g., a full-length AK155 alpha receptor subunit (e.g., SEQ ID NO: 1), an AK155 receptor, or a polypeptide comprising an AK155 binding domain of an AK155 alpha subunit).

One type of assay that can be used is a direct binding assay, which measures the amount of candidate therapeutic agent that can bind to a AK155 receptor or to a polypeptide that has an AK155 binding domain. Another type of assay that can be used to screen candidate therapeutic agents is to carry out a displacement binding assay with a labeled AK155 receptor ligand (e.g. a labeled AK155 molecule) in the presence of a candidate therapeutic agent. In certain embodiments, the assays are carried out using cells that express an AK155 receptor. Cells that have been transfected with the subunits of an AK155 receptor can be used in these assays. A candidate therapeutic agent that decreases the amount of labeled AK155 receptor ligand that is bound to a AK155 receptor (e.g., on the surface of a cell or cell membrane), or a polypeptide that has a AK155 ligand binding domain, is of interest for future screening for its ability to modulate AK155-mediated and AK155-receptor mediated diseases and conditions in vivo.

These assays can also be carried out using labeled candidate therapeutic agents which are then incubated with a polypeptide that has a AK155 ligand binding domain. Labels include radioisotopes, immunochemicals, fluorophores, and the like. Those of skill in the art will recognize a variety of ways of separating the bound labeled candidate therapeutic agent from the free labeled candidate therapeutic agent. The affinity of the labeled candidate therapeutic agent for a AK155 ligand binding domain can be calculated using standard ligand binding methods.

E. Gene Reporter Assays.

Also provided by the invention are methods for pre-screening candidate therapeutic agents as in a reporter gene assay. The methods can involve transfecting a cell that expresses an AK155 receptor and contains an AK155 responsive transcription factor (e.g., STAT3) with a reporter gene expression cassette. The reporter gene expression cassette contains an AK155 responsive transcription factor binding site (e.g., a STAT3 response element, a IFN-γ-activated-sequences (GAS), etc.), such that when AK155 responsive transcription factor binds to the transcription factor binding site that transcription of the reporter gene takes place. In certain embodiments, the promoter and response element are operably linked to a reporter gene that, when expressed, produces a readily detectable product. A variety of reporter gene plasmid systems are known, such as the chloramphenicol acetyltransferase (CAT) and β-galactosidase (e.g., bacterial LacZ gene) reporter systems, the firefly luciferase gene (See, e.g., Cara et al., (1996) J. Biol. Chem., 271:5393-5397), the green fluorescence protein (see, e.g., Chalfie et al. (1994) Science 263:802) and many others. Examples of reporter plasmids are also described in U.S. Pat. No. 5,071,773. Selectable markers which facilitate cloning of the vectors of the invention are optionally included. Sambrook and Ausubel, both supra, provide an overview of selectable markers. For mammalian host cells, preferred transfection methods include, for example, calcium phosphate precipitation (Chen and Okayama (1988) BioTechniques 6:632-638), DEAE-dextran, and cationic lipid-mediated transfection (e.g., Lipofectin) (see, e.g., Ausubel, supra.).

The cell containing the AK155 receptor is contacted with a candidate therapeutic agent. For example, a cell that contains a reporter gene construct and the AK155 receptor can be grown in the presence and absence of a candidate therapeutic agent. Cells contacted with AK155 or with a candidate therapeutic agent that activate an AK155 receptor will exhibit reporter gene expression. If the candidate therapeutic agent is an AK155 receptor antagonist, then an increase in reporter gene expression seen with AK155 would be inhibited.

Those of skill in the art will recognize a wide variety of cells that may be used in the methods of the present invention. The cells may be, but are not limited to, primary cultures of cells, transformed cells, neoplastic cells, and nontransformed cells. Cell lines that may be used in the gene expression assay include Colo-205 (colon carcinoma), SW-403 (colon carcinoma), Lovo (colon carcinoma), HaCaT (keratinocytes, IL-20 reactive) cells and the like.

F. Assays for mRNAs Modulated by AK155 or an AK155 Receptor.

Another method of the present invention involves assaying for the amount of mRNA of a gene whose presence is increased or decreased in response to an AK155 binding to an AK155 receptor or in response to the activation of an AK155 receptor by another molecule or mechanism. Examples of such a gene include, without limitation, genes that encode IL-8, IL-10, ICAM-1, ICAM-2 and B7-H1 (GenBank Accession No. XM016319).

Briefly, cells are incubated in the presence and absence of a candidate therapeutic agent. After the incubation, RNA from the cells is isolated. The presence of the mRNA of an AK155-regulated gene or AK155-receptor activated gene can then be detected through methods known to those of skill in the art. For example, one can detect the RNA through reverse transcription followed by PCR. Alternatively, the RNA can be electrophoresed, blotted, and hybridized with an appropriate probe directed towards the gene of interest. Other methods, such as RNAse protection, are known in the art for detecting RNA sequences of interest. Candidate therapeutic agents that block AK155 mediated transcription events are useful as antagonists of AK155 mediated processes.

G. Assays for Polypeptides Modulated by AK155 or an AK155 Receptor Mediated Process.

Screening assays can also be carried out by measuring the effects of candidate therapeutic agents on the levels, subcellular localization, and post-translational modification of polypeptides associated with the up- or down-regulation of an AK155 receptor (e.g., phosphorylated STAT3). For example, immunoblots and immunoassays can be used to assay for the effect of candidate therapeutic agents on molecules that are upregulated by AK155 binding to its receptor, including, IL-8, IL-10, ICAM-1, ICAM-2 and B7-H1, and phosphorylated STAT3. In addition, candidate therapeutic agents can be assayed for their ability to decrease or increase the phosphorylation state of STAT3 and the DNA binding activity of STAT3 (e.g., through gel-shift assays using nuclear extracts). Both of those processes are increased by the binding of AK155 to a cell that expresses an AK155 receptor (see e.g., Example section).

H. Candidate Therapeutic Agents.

A “candidate therapeutic agent,” is a compound that is being tested for its usefulness in the treatment of an AK155-mediated disorder or condition, or is being tested in a ligand binding assay or a gene activation assay. Candidate therapeutic agents include anti-inflammatory agents. The candidate therapeutic agent can be a naturally occurring compound, one that is artificially synthesized, or one that is made by a combination these methods. Candidate therapeutic agents include, without limitation, small organic molecules (e.g., organic molecules of less than 5000 Da, organic molecules of less than under 1000 Da, organic molecules of less than 500 Da, etc.), antibodies, antibody fragments, polypeptides, peptides, etc. Essentially any chemical compound can be pre-screened as an AK155 or AK155 receptor candidate therapeutic agent in the assays of the invention. Thus, candidate therapeutic agents include the compounds that are “suspected of inhibiting receptor activation induced by binding of AK155 to the receptor.” It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

A candidate therapeutic agent “modulates” an AK155 or an AK155 receptor if the candidate therapeutic agent increases or decreases the ability of an AK155 polypeptide or AK155 receptor to act in a selected system. Such selected systems include, but are not limited to inflammation, angiogenesis, cell growth (cancer), Crohn's disease, inflammatory bowel disease, colon carcinoma, Hodgkin's disease, and the assays described herein (e.g., direct binding assays, transcription based assays, etc.). Subjects or assays that are treated with a candidate therapeutic agent are compared to control samples without the candidate therapeutic agent, to examine the extent of inhibition or activation of AK155-mediated processes. Control samples (untreated with a candidate therapeutic agent) are assigned a relative AK155 activity (or AK155 receptor) value of 100. Inhibition of AK155 activity is achieved when the AK155 activity value of the candidate therapeutic agent sample relative to the control is about 85, preferably 75, more preferably 50, and still more preferably 25. Activation is achieved when the AK155 activity value of the test sample relative to the control is about 110, preferably 125, still more preferably 150, and even more preferably 200.

In some preferred embodiments, high throughput screening methods are used to test a combinatorial library that contains a large number of potential therapeutic compounds (potential modulator compounds). In some embodiments, compounds that can be dissolved in aqueous or organic (e.g., DMSO-based) solutions are used. The assays are designed to screen large chemical libraries by automating the assay steps and providing compounds from any convenient source to assays, which are typically run in parallel (e.g., in microtiter formats on microtiter plates in robotic assays). “Combinatorial chemical libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka (1991) Int. J. Pept. Prot. Res. 37:487-493 and Houghton et al. (1991) Nature 354:84-88). Other chemistries for generating chemical diversity libraries can also be used. Such chemistries include, but are not limited to: peptides (PCT Publication No. WO 91/19735), encoded peptides (PCT Publication WO 93/20242), random bio-oligomers (PCT Publication No. WO 92/00091), benzodiazepines (U.S. Pat. No. 5,288,514), diversomers such as hydantoins, benzodiazepines and dipeptides (Hobbs et al. (1993) Proc. Nat. Acad. Sci. USA 90:6909-6913), vinylogous polypeptides (Hagihara et al. (1992) J. Amer. Chem. Soc. 114:6568), nonpeptidal peptidomimetics with β-D-glucose scaffolding (Hirschmann et al. (1992) J. Amer. Chem. Soc. 114:9217-9218), analogous organic syntheses of small compound libraries (Chen et al. (1994) J. Amer. Chem. Soc. 116:2661), oligocarbamates (Cho et al. (1993) Science 261:1303), and/or peptidyl phosphonates (Campbell et al. (1994) J. Org. Chem. 59:658), nucleic acid libraries (see, Ausubel, Berger and Sambrook, all supra), peptide nucleic acid libraries (see, e.g., U.S. Pat. No. 5,539,083), antibody libraries (see, e.g., Vaughn et al. (1996) Nature Biotechnology, 14(3):309-314 and PCT/US96/10287), carbohydrate libraries (see, e.g., Liang et al. (1996) Science, 274:1520-1522 and U.S. Pat. No. 5,593,853), small organic molecule libraries (see, e.g., benzodiazepines, Baum C&EN, Jan 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, 5,288,514, and the like).

III. In vivo Testing of Candidate Therapeutic Agents.

The term “administering” refers to the method of contacting a compound with the subject. Modes of “administering,” may include but are not limited to, methods that involve contacting the compound intravenously, intraperitoneally, intranasally, transdermally, topically, via implantation, subcutaneously, parentally, intramuscularly, orally, systemically, and via adsorption. The candidate therapeutic agent can be formulated as a pharmaceutical composition in the form of a syrup, an elixir, a suspension, a powder, a granule, a tablet, a capsule, a lozenge, a troche, an aqueous solution, a cream, an ointment, a lotion, a gel, or an emulsion.

Several embodiments of methods can be used to test the efficacy of a candidate therapeutic agent to modulate one or AK155-mediated processes (e.g., inflammation). Preferably, the candidate therapeutic agent will act as an antagonist and decrease deleterious effects of AK155 in a system (i.e., decrease inflammation or decrease the symptoms or severity of a disease or condition). The effectiveness of a candidate therapeutic agent can be ascertained using the assays described herein, as well as the qualitative and quantitative observations of a patient or treating physician.

IV. Methods for Treating or Preventing Ak155-Mediated and AK155-Receptor Mediated Diseases and Conditions.

AK155-mediated and AK155-receptor mediated diseases and conditions, such as inflammation, angiogenesis, cell growth (cancer), Crohn's disease, inflammatory bowel disease, colon carcinoma, Hodgkin's disease, etc.), can be treated with therapeutic agent(s) identified using the methods described herein. The candidate therapeutic agents can be pre-screened using the methods described herein. The candidate therapeutic agent is prepared as a pharmaceutical composition and is administered to a subject suffering from an AK155-mediated or an AK155-receptor mediated disease or condition.

A. Disease Conditions.

The invention is contemplated for use in the treatment of the disease states such as inflammation, skin conditions, allergies, and cancer.

i. Cancer, tumors, and angiogenesis. The invention is contemplated to be useful for controlling cancer cells, tumors, and other proliferating cells. Cytokines can be used for the treatment of various tumors. IL-2 administration provokes an increase in number of T cells, B cells, and NK cells, where these cells have antitumor activity. IL-2 treatment may be successful in treating melanoma and renal cell carcinoma. Interferon-α treatment, which increases the cytotoxic effect of NK cells, can be used to treat melanoma, renal carcinoma, lymphomas, and hairy cell leukemia (Abbas, et al. (2000) Cellular and Mol. Immunol. 4^{th} ed., W. B. Saunders Co., New York, p. 400).

IL-12 treatment can inhibit tumor growth, metastasis, and angiogenesis. Eradication of metastasis is initiated by IL-12's induction of T cells. Inhibition of angiogenesis by IL-12 requires the participation of IFN-γ which, in turn, stimulates IP-10, a CXC chemokine. IP-10, in turn, induces T cells to inhibit angiogenesis (Pertl, et al., J. Immunol. 166, 6944 (2001)). IL-10 can have an antitumor effect, where administration of IL-12 together with IL-10 can have an additive antitumor effect (Berman, et al., J. Immunol. 157, 231 (1996)). IL-10 has been found to have an antitumor effect in animal studies of mastocytoma, breast cancer, melanoma, prostate cancer, and colon carcinoma. It is interesting to note that treatment with the variant of IL-10 of Epstein-Barr virus does not have an anti-tumor effect, but instead has a pro-tumor effect (Moore, et al. (2001) Annu. Rev. Immunol. 19:683).

Interferon-α plus zidovudine (inhibits DNA replication) has been found to produce dramatic results in the treatment of adult T-cell leukemia-lymphoma (Gill, et al. (1995) New Engl. J. Med. 332:1744). IL-2 with IFN-α-2a has been found to produce a measurable response in patients with metastatic renal-cell carcinoma (Negrier, et al. (1998) New Engl. J. Med. 338:1272).

The above-described studies involve systemic administration of cytokines, while the following study reveals a mode of highly localized cytokine treatment. The C—C chemokine family attracts monocytes and lymphocytes, while C—X—C chemokines attract neutrophils and lymphocytes. For example, RANTES, a C—C chemokine, attracts T cells, NK cells, monocytes, eosinophils, basophils, and dendritic cells. Use of chemokines in anti-cancer therapy is illustrated by a bifunctional protein composed of RANTES linked to an antibody recognizing a tumor-specific antigen (RANTES-antibody). The antibody domain targets the bifunctional protein to cancer cells, while the chemokine moiety attracts immune cells which destroy the targeted cancer cell (Challita-Eid, et al. (1998) J. Immuol. 161:3729).

ii. IgE-dependent disease. The contemplated invention is expected to be useful for treating IgE-dependent disease conditions, such as asthma, anaphylaxis, and allergic rhinitis (Salvi and Babu (2000) New Engl. J. Med. 342:1292; (Teran (2000) Immunol. Today 21:235; Marone (1998) Immunol. Today 19:5; Corrigan (1999) Clin. Exp. Immunol. 116:1).

Asthma is characterized by three features: intermittent and reversible airway obstruction, airway hyperresponsiveness, and airway inflammation (Galli (1997) J. Exp. Med. 186:343). Asthma involves the following series of events. Inhaled allergens encounter dendritic cells (allergen presenting cells; APCs) that line the airway. The dendritic cells then migrate to lymph nodes, where they present antigen to T cells. Contact of the dendritic cells with the T cells activates the T cells, and once activated, the T cells produce IL-4 and IL-13 (which act on B cells to promote IgE production) and IL-5 (which recruits eosinophils) (Jaffar, et al. (1999) J. Immunol. 163:6283).

B cells reside in lymph nodes. Two signals are required to provoke B cells to secrete IgE: (1) IL-4 (or IL-13) contact with B cells; and (2) T cell contact with B cells. The occurrence of both of these signals provokes the B cells to produce IgE. The IgE, in turn, circulates in the blood, where it may bind FcεRI of mast cells and basophils, provoking the mast cells and basophils to release various inflammatory agents and toxins. Mast cells can produce IL-1, IL-2, IL-3, IL-4, IL-5, granulocyte-macrophage stimulating factor, IFN-γ, and TNF-α, histamine, leukotrienes, and toxic oxygen. Histamine and leukotrienes can provoke smooth muscles to contract, resulting in airway obstruction. IL-5 can recruit eosinophils, and once recruited, the eosinophils may produce “major basic protein,” a protein that can directly damage the airways (Plager, et al. (1999) J. Biol. Chem. 274:14464). The eosinophils produce leukotrienes, which can provoke the airways to contract.

“Recruitment” of eosinophils means provoking or inducing the migration of eosinophils to migrate from the bloodstream to other locations, such as the airway. Recruitment may involve activation of integrin on the eosinophil surface, where activation is provoked by various cytokines. These cytokines may include eotaxin, RANTES, macrophage inflammatory protein-1α, and monocyte chemotactic protein 1 (Busse and Lemanske (2001) New Engl. J. Med. 344:350).

Environmental allergens initiate the pathway leading to the production of IgE by B cells. These allergens also are used for the cross-linking of IgE/FcεRI complexes residing on the surface of mast cells, where the cross-linking results in mast cell activation. Most cases of asthma occur in people who are hypersensitive to specific environmental allergens, such as dust mite allergen, cockroach allergen, pollen, and molds (Barnes (1999) New Engl. J. Med. 341:2006). In humans, IgE is the main or only type of immunoglobin (Ig) that mediates airway hypersensitivity (Galli (1997) J. Exp. Med. 186:343). In fact, there is a strong correlation between serum IgE levels and asthma. IgE is elevated in patients with bronchial asthma and allergic rhinitis (Zuberi, et al. (2000) J. Immunol. 164:2667). Mast cells express receptors (FcεRI) that bind the constant region of IgE antibodies. Injections of recombinant antibodies against IgE have been used to treat asthma. Here, the anti-IgE binds to IgE in the body, and competitively prevents this IgE from binding to Fc receptors (Barnes (1999) New Engl. J. Med. 341:2006). Anti-IgE treatment in humans also can result in the down-regulation of FcεRI, as determined by studies of basophils and other cells (Saini, et al. (1999) J. Immunol. 162:5624).

When a mast cell bearing bound IgE molecules encounters an antigen recognized by the bound IgE molecule, the antigen binds, resulting in the mast cell secreting histamine, proteases, prostaglandins, leukotrienes, toxic oxygen, and cytokines. In this situation, the allergen cross-links IgE molecules that are bound to FcεRI, resulting in activation of the mast cell (Kita, et al. (1999) J. Immunol. 162: 6901).

The airways of asthma patients contain accumulations of mast cells, but also of T cells (Th2 type), eosinophils, basophils, and macrophages. Macrophages express FcεRIIB (low affinity IgE receptor), where binding of IgE plus allergen can stimulate the macrophage to release prostaglandins, toxic oxygen, and cytokines (Ten, et al. (1999) J. Immunol. 163:3851).

IL-10 may be involved in allergic diseases, such as asthma, since IL-10 can inhibit cytokine production by eosinophils, can inhibit cytokine production by mast cells, and can inhibit airway neutrophilia and eosinophilia induced by antigenic challenge (Moore, et al. (2001) Annu. Rev. Immunol. 19:683; Zuany-Amorim, et al. (1995) J. Clin. Invest. 95:2644; Stampfli, et al. (1999) Am. J. Respir. Cell Mol. Biol. 21:586).

iii. Inflammatory diseases of the gut. The contemplated reagent is expected to be of use for the treatment of inflammatory diseases of the gut, such as inflammatory bowel disease, Crohn's disease (Beutler (2001) Immunity 15:5; Targan et al. (1997) New Engl. J. Med. 337:1029), colitis (Simpson, et al. (1998) J. Exp. Med. 187:1225), and celiac disease. IL-10 is likely to contribute to inflammatory bowel disease, as IL-10-deficient mice exhibit this disease and administration of IL-10 can prevent it (Kuhn, et al. (1993) Cell 75:263; Moore, et al. (2001) Annu. Rev. Immunol. 19:683).

iv. Autoimmune diseases. The contemplated reagent is also expected to be of use for treatment of autoimmune diseases, such as multiple sclerosis, diabetes mellitus, systemic lupus erythematosus (SLE), Sjogren's syndrome, scleroderma, polymyositis, autoimmune thyroid disease, autoimmune gastritis and pernicious anemia, and autoimmune hepatitis (Bradley, et al. (1999) J. Immunol. 162:2511; Stott, et al. (1998) J. Clin. Invest. 102:938; The Autoimmune Diseases, 3rd ed. (1998) Ed. by N. R. Rose and I. R. Mackay, Academic Press, San Diego, Calif.).

SLE may be treated with anti-IL-10 (Ishida, et al. (1994) J. Exp. Med. 179:305; Llorente, et al. (1998) J. Exp. Biol. 181:839; Moore, et al. (2001) Annu. Rev. Immunol. 19:683; Llorente, et al. (2000) Arthritis Rheum. 43:1790). IL-10 is thought to contribute to systemic lupus erythematosus because of the high expression of IL-10 in this disease (Moore, et al. (2001) Annu. Rev. Immunol. 19:683).

v. Immune disease of the nervous system. The invention is contemplated to be useful for the treatment of diseases of the central and peripheral nervous systems, such as multiple sclerosis, perivenous encephalomyelitis, acute necrotizing hemorrhagic leukoencephalomyelitis, Guillain-Barre Syndrome, demyelinating neuropathy, and the POEMS Syndrome. Neutralization of IL-10 increases the severity of experimental autoimmune encephalomyelitis (EAE), an animal model for multiple sclerosis, while IL-10 treatment may inhibit EAE (Crisi, et al. (1995) Eur. J. Immunol. 23:3035; Moore, et al. (2001) Annu. Rev. Immunol. 19:683). It is interesting to point out the correlation between infection with Epstein-Barr virus and multiple sclerosis (Wandinger, et al. (2000) Neurology 55:178), the production of a viral version of IL-10 by Epstein-Barr virus, and the array of similar and different effects of human IL-10 and viral IL-10 (Moore, et al. (2001) Annu. Rev. Immunol. 19:683).

vi. Inflammatory and immune-related disease of the skin. The contemplated reagent is expected to be useful for the treatment of disease states of the skin such as psoriasis, systemic lupus erythematosus (Tsokos and Liossis (1999) Immunol. Today 20:119), vitiligo, dermatitis herpetiformis, alopecia, atopic eczema, and atopic dermatitis (Robert and Kupper (1999) New Engl. J. Med. 341:1817), as well as autoimmune skin diseases that are organ specific, such as pephigus vulgaris, bullous pemphigoid, and pemphigus foliaceus (Davidson and Diamond (2001) New Engl. J. Med. 345:340). The contemplated reagent is also expected to be useful for enhancing the healing of chronic ulcers. The expected use of the invention for treatment of skin conditions is supported by the presence of AK155 receptor in keratinocytes (See, e.g., Example 5).

Psoriasis is a skin disease involving hyperproliferation of keratinocytes, and an influx of T cells, neutrophils, macrophages, and dendritic cells. These T cells include skin homing T cells, that is, T cells that had passed through the blood vessel wall and left the bloodstream to migrate to the skin. Keratinocytes and antigen presenting cells (APCs) in the skin activate the T cells, where the activated T cells secrete growth factors and cytokines which, in turn, provoke keratinocyte growth (Bos and De Rie, Immunol. Today 20, 40 (1999)). There is some thought that CD4⁺ T cells help initiate skin lesions, while CD8⁺ T Cells are responsible for the persistence of the lesions (Robert and Kupper (1999) New Engl. J. Med. 341:1817). The study of psoriasis is sometimes divided into an examination of factors that produce keratinocyte hyperproliferation and factors that produce inflammation.

Psoriasis appears to be partly dependent on interferon-γ (IFN-γ.) IFN-γ is produced by T cells, where it is produced by CD4⁺ T cells, CD8⁺ T cells (Szabo, et al. (1998) J. Invest. Dermatol. 111:1072), and mast cells (Ackermann, et al. (1999) Br. J. Dermatol. 140:624). Studies of keratinocytes have shown that T cells isolated from psoriatic lesions secrete large amounts of interferon-γ. The study also revealed that T cells promote keratinocyte proliferation by an IFN-γ dependent pathway (Hong, et al. (1999) J. Immunol. 162:7480).

Psoriasis may be dependent on IL-2, as revealed by the following three studies: (1) Treating human white blood cells with IL-2 produced psoriasis-like symptoms. The study involved biopsies of human skin, and human white blood cells treated with IL-2, where the human skin was transplanted on mice and the cells were injected in the mice. The study demonstrated that IL-2 treatment was required for the production of psoriatic skin (Wrone-Smith and Nickoloff (1996) J. Clin. Inv. 98:1878); (2) Treating human patients with a fusion protein consisting of diphtheria toxin (a poison) linked to IL-2, resulted in an improvement of the disease (Granstein, J. Clin. Invest. 98, 1695 (1996); Gottleib, et al. (1995) Nature Medicine 5:442); and

(3) Cyclosporin treatment of psoriasis patients resulted in a decrease in the number of T cells (where these T cells bore an IL-2 receptor) and in an improvement of the disease (Gottlieb, et al. (1992) J. Invest. Dermatol. 98:302).

Psoriasis may be dependent on IL-12. A mouse model of psoriasis was produced by injecting T cells into mice. The T cells were genetically deficient in IFN-γ, thus enabling detection of IFN-γ-independent pathways of psoriasis. The T cell injection resulted in psoriasis. This mouse model of psoriasis closely resembled human psoriasis, as it resulted in down growths of epidermis into dermis, called “elongation of rete pegs.” The disease was prevented by injections of anti-IL-12 (Hong, et al. (2001) J. Immunol. 166:4765).

Psoriasis may be dependent on CCL20. Studies of skin homing have revealed that keratinocytes produce a CC cytokine, namely, CCL20 (macrophage inflammatory protein-3α). This cytokine binds to a receptor on T cells, called CCR6. Expression of both of these proteins is greatly elevated in psoriatic skin, as compared to normal skin (Homey, et al. (2001) J. Immunol. 164:6621).

Inflammatory skin diseases may be dependent on the chemokine CCL27, also known as CTACK. CCL27 is constitutively produced by keratinocytes, and can be induced by TNF-α and IL-1β. CCL27's receptor is CCR10. CCR10 occurs on T cells, as well a number of other cells (Homey, et al., J. Immunol. 164, 3465 (2000)). CCL27 is detected only in the skin, as revealed by studies of humans and mice (Morales, et al. (1999) Proc. Natl. Acad. Sci. USA 96:14470). CCL27 can attract a subset of T cells, namely, the CLA⁺ memory T cells. Molecules used for the homing of T cells to the skin are discussed. About 90% of the T cells in inflammatory skin lesions express CLA, while under 5% of the T cells in non-skin inflamed sites express CLA. CLA is a membrane-bound protein that is a ligand for E-selectin (membrane-bound protein of epithelial cells of blood vessels). Interaction between CLA and E-selectin may be critical for recruiting T cells to sites of skin inflammation (Morales, et al. (1999) Proc. Natl. Acad. Sci. 96:14470). E-selectin is upregulated during inflammation (Tietz, et al. (1998) J. Immunol. 161:963). Another selectin, called P-selectin, also occurs on endothelial cells. T cells from psoriatic skin express ligands for both E- and P-selectin (Chu, et al. (1999) J. Immunol. 163:5086). Studies with mice demonstrated that E- and P-selectin have functions that are quite similar (Tietz, et al. (1998) J. Immunol. 161:963).

In addition to CLA, another membrane-bound protein of the T cell is used in T cell homing to the skin. LFA-1 is a membrane-bound protein of T cells. LFA-1 is a member of the integrin family of proteins. For T cells to bind to endothelial cells of a blood vessel, LFA-1 must first be activated. Activation appears to be dependent on chemokine receptors on the surface of the T cell (Stein, et al. (2000) J. Exp. Med. 191:61). Once LFA-1 is activated, it binds to ICAM (ICAM is an extracellular protein on the vascular endothelium). Clinical studies have shown that antibodies to LFA-1 can be used to treat psoriasis (Weitz-Schmidt, et al. (2001) Nature Medicine 7:687; Granstein (2001) New Engl. J. Med. 345:284; Gottlieb, et al. (2000) J. Am. Acad. Dermatol. 42:428).

Psoriasis may be triggered by bacterial antigens (Granstein (1996) J. Clin. Invest. 98:1695; Robert and Kupper (1999) New Engl. J. Med. 341:1817; Chu, et al. (1999) J. Immunol. 163:5086). Bacterial products, such as lipopolysaccharides, can activate white blood cells through the Toll-like receptor family of proteins (TLRs). The activated white blood cells, in turn, can release cytokines that recruit CLA⁺ T cells to the skin. Alternatively, bacterial products may bind to MHC II and consequently serve to activate T cells (Travers, et al. (1999) J. Clin. Invest. 104:1181). In this scenario, the bacterial product is called a “superantigen.” Note that MHC II, when complexed with an antigen, serves to activate the T cell receptor.

It will be apparent from the above commentary that the contemplated invention may be used for treating inflammatory conditions by interfering with the action of IFN-γ, IL-2, IL-12, CCL27, CCL20, T cell receptor, the Toll-like receptors, and T cell homing proteins, such as LFA-3.

vii. Immune-related diseases of muscle. The above mentioned reagent is expected to be useful for autoimmune diseases of the muscle, such as myasthenia gravis (Balasa and Sarvetnick (2000) Immunol. Today 21:19; Sempowski, et al. (2001) J. Immunol. 166:2808), Lambert-Eaton myasthenic syndrome, polymyositis, and idiopathic inflammatory myopathy (The Autoimmune Diseases, 3rd ed. (1998) Ed. by N. R. Rose and I. R. Mackay, Academic Press, San Diego, Calif.).

viii. Transplant-related immune diseases. The invention is contemplated to be of use for treatment of transplant rejection and graft versus host disease (GVHD) (Blazar, et al. (1997) Immunol. Revs. 157:79). Studies with animals revealed that administration of IL-10 increased survival of various grafts and reduced GVHD-associated lethality (Moore, et al. (2001) Annu. Rev. Immunol. 19:683). In mice, Epstein-Barr virus IL-10 can inhibit the rejection of transplanted organs (Qin, et al. (1996) J. Immunol. 156:2316-2323; Suzuki, et al. (1995) J. Exp. Med. 182:477-486).

ix. Systemic inflammation. The invention is contemplated to be useful for the treatment of systemic inflammatory response, as may occur in, septicaemia, septic shock, severe trauma, burns, and reperfusion injury. IL-10 has a protective role in endotoxemia (Pajkrt, et al. (1997) J. Immunol. 158:3971), while IL-10-deficient mice have a dramatically increased susceptibility to lipopolysaccharide (Berg, et al. (1995) J. Clin. Inv. 96:2339; Moore, et al. (2001) Annu. Rev. Immunol. 19:683).

x. Infection. It is contemplated to use the invention for the treatment of bacterial, fungal, and protozoal infections. A typical response to infections may involve recognition of the microbe or microbial products by macrophages, neutrophils, or dendritic cells. Macrophages may respond by producing various cytokines, i.e., IL-12, TNF, IL-1, and IL-18. The combination of IL-12, IL-1, and TNF, in turn, stimulates NK cells to produce interferon-γ (IFN-γ). IFN-γ, in turn, induces macrophages to produce toxic oxygen, to engage in phagocytosis, and induces infiltration by macrophages and neutrophils. IL-12 and IFN-γ provoke T cells to mount a further response to microbes (Moore, et al., Annu. Rev. Immunol. 19, 683 (2001)). Resistance to infection can be improved by reducing IL-10 levels, as shown by studies of IL-10-deficient mice and by treatment with anti-IL-10 (Dai, et al., J. Immunol. 158, 2259 (1997); Wagner, et al. (1994) Infect. Immun. 62:2345; Vazquez-Torres, et al. (1999) Infect. Immun. 67:670).

B. Pharmaceutical Compositions.

Accordingly, the present invention provides pharmaceutical compositions comprising a pharmaceutically acceptable carrier or excipient and a candidate therapeutic agent.

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavoring agents, binders, preservatives, tablet disintegrating agents, or an encapsulating material.

In powders, the carrier is a finely divided solid which is in a mixture with the finely divided active component. In tablets, the active component is mixed with the carrier having the necessary binding properties in suitable proportions and compacted in the shape and size desired.

The powders and tablets preferably contain from 5% or 10% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 0.1 mg to 1000 mg, preferably 1.0 mg to 100 mg according to the particular application and the potency of the active component. The composition can, if desired, also contain other compatible therapeutic agents.

C. Treatment Regime Using Candidate Therapeutic Agents.

The present invention also provides methods of promoting AK155 activity (or AK155 receptor activity) or blocking AK155 antagonist activity (or AK155-receptor activity) in a cell. For example, a cell can be contacted with an AK155 or AK155-receptor-inhibiting amount of a compound or composition above. An AK155-inhibiting amount can be readily determined using the assays described herein. Typically, the amount or concentration of compound required to achieve EC₅₀ will be considered an AK155 activating or an AK155 antagonist inhibiting amount. Candidate therapeutic agents are especially useful in the treatment of inflammation.

In another aspect, the present invention provides methods of treating conditions modulated by an AK155 or an AK155-receptor in a host, by administering to the host an effective amount of a compound or composition provided above. In therapeutic applications, the compounds of the present invention can be prepared and administered in a wide variety of oral and parenteral dosage forms. Thus, the compounds of the present invention can be administered by injection, that is, intravenously, intramuscularly, intracutaneously, subcutaneously, intraduodenally, or intraperitoneally. Also, the compounds described herein can be administered by inhalation, for example, intranasally. Additionally, the compounds of the present invention can be administered transdermally.

A variety of conditions are modulated, at least in part, by an AK155 or an AK155 receptor, including inflammation, angiogenesis, cell growth, etc. The compounds utilized in the pharmaceutical method of the invention are administered at the initial dosage of about 0.001 mg/kg to about 100 mg/kg daily. A daily dose range of about 0.1 mg/kg to about 10 mg/kg is preferred. The dosages, however, may be varied depending upon the requirements of the patient, the severity of the condition being treated, and the compound being employed. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. For convenience, the total daily dosage may be divided and administered in portions during the day, if desired.

Typically, the host or subject in each of these methods is human, although other animals can also benefit from the foregoing treatments.

### EXAMPLES

I. General Methods.

Some of the standard methods are described or referenced, e.g., in Maniatis, et al. (1982) Molecular Cloning, A Laboratory Manual, Cold Spring Harbor Laboratory, Cold Spring Harbor Press; Sambrook and Russell (2001) Molecular Cloning, 3^{rd} ed., Cold Spring Harbor Laboratory Press, Cold Spring Harbor, N.Y.; Wu (1993) Recombinant DNA, Vol. 217, Academic Press, San Diego, Calif.; Innis, et al. (eds.) (1990) PCR Protocols: A Guide to Methods and Applications, Academic Press, N.Y. Standard methods are also found in Ausbel, et al (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification such as ammonium sulfate fractionation, column chromatography, electrophoresis, isoelectric focusing, centrifugation, and crystallization, are described (Coligan, et al. (2000) Current Protocols in Protein Science, Vol. 1, John Wiley and Sons, Inc., New York). Chemical analysis, chemical modification, post-translational modification, and glycosylation of proteins is described (Coligan, et al. (2000) Current Protocols in Protein Science, Vol. 2, John Wiley and Sons, Inc., New York). The production, purification, and fragmentation of polyclonal and monoclonal antibodies is described (Coligan, et al. (2001) Current Protcols in Immunology, Vol. 1, John Wiley and Sons, Inc., New York; Harlow and Lane (1999) Using Antibodies, Cold Spring Harbor Laboratory Press, Cold Spring Harbor, N.Y.); Harlow and Lane (1988) Antibodies A Laboratory Manual, Cold Spring Harbor Laboratory, Cold Spring Harbor, N.Y.).

Standard techniques for characterizing ligand/receptor interactions are described (Coligan, et al. (2001) Current Protcols in Immunology, Vol. 4, John Wiley and Sons, Inc., New York).

Cell culture techniques are described in Doyle, et al. (eds.) (1994) Cell and Tissue Culture: Laboratory Procedures, John Wiley and Sons, NY. FACS analysis is described in Melamed, et al. (1990) Flow Cytometry and Sorting Wiley-Liss, Inc., New York, N.Y.; Shapiro (1988) Practical Flow Cytometry Liss, New York, N.Y.; and Robinson, et al. (1993) Handbook of Flow Cytometry Methods Wiley-Liss, New York, N.Y.

Reagents for fluorescent labeling of proteins, nucleic acids, lipids, carbohydrates, and cells are from Molecular Probes, Inc. (Eugene, Oreg.). These reagents include those that modify thiols, amines, hydroxylated molecules, fluorescent biotin derivatives, and photoreactive reagents.

The following examples are offered to illustrate, but not to limit the claimed invention.

### Example 1

The experiments described in this example demonstrate methods that may be used for cloning both the α- and β-subunits of the AK155 receptor. The experiments also describe methods by which cells that already express the β-subunit of the AK155 receptor (IL-10Rβ; IL-10R2) are transfected with the α-subunit of the AK155 receptor subunit (IL-20Rα; IL-20R1), and finally, the experiments reported in this example demonstrate how the AK155 receptor is expressed in a cell following cloning and transfection.

Using methods outlined in Balbas and Bolivar (1990) Methods in Enzymology 185: 14-37, the α subunit of the AK155 receptor was cloned into any one of a number of preferred vectors for expression. Vectors used for expression of genes in mammalian cells include pME-X, pCD-SRa, pCD, pDNA1, etc. Typically these vectors contain a promoter such as that is active in mammalian cells, stop and poyadenylation signals, and a drug-selection marker such as neomycin-resistance. In addition these vectors contain antibiotic resistance genes and an origin of replication for propagation in bacteria (see Okayama and Berg (1985) Mol. Cell Biol. 5:1136-1142).

Cells were transfected using liposomes that were complexed with expression vectors for an AK155 alpha subunit (FLAG-tagged CPNM 1; CPNM1-FLAG) and an AK155 β subunit (IL-10Rβ). Briefly, cells were cultured in DME with 5% fetal calf serum (FCS) until semiconfluent. Fugene 6® (Roche Molecular Biochemicals, Indianapolis, Ind., Cat. No. 1814443) was diluted 1/40 in DME without serum and added dropwise to plasmid DNA in DME without serum at a ratio of 2 μl undiluted Fugene 6®:1 μg DNA. Plasmid/lipid solution was incubated for 15 min at room temperature and added dropwise to cells after changing media to DME without serum.

Alternatively, Ba/F3 cells (Murine B cell line; Palacios and Steinmetz (1985) Cell 41:727-734), which also expresses a β-subunit of the AK155 receptor, were infected with retrovirus containing FLAG-CPNM1 (AK155 α subunit) using retronectin-mediated infection. In certain cases, the cells were also transfected with retrovirus containing an AK155 beta subunit (hIL-10Rβ). Briefly, 60 mm petri dishes were coated with 2 ml of a 30-40 μg/ml dilution of retronectin (Takara Cat no T100b) in water for 2 hrs at room temperature. Subsequently, the retronectin solution was removed and the petri dishes blocked with PBS/2% BSA for 30 minutes at room temperature. Plates were washed with culture medium and cells (100.000) were applied. After 10 minutes, retrovirus containing supernatant harvested from packaging cells was added (Kitamura (1998) Int J Hematol 67:351, Kitamura (2000) Methods Mol. Biol. 134:143, Kitamura (1995) Proc Natl Acad Sci USA 92:9146).

The expression of the CPNM1-FLAG subunit was monitored using a biotinylated anti-FLAG (Sigma) mAb followed by Streptavidin-PE. Streptavidin-PE Cells were analyzed by fluorescence-activated-cell-sorting (FACS) and positive cells were sorted. An anit-hIL-20Rβ mAb was used to monitor the expression of hIL-20Rβ.

### Example 2

The experiments reported in this example demonstrate how the expression of chemokines, chemokine receptors and cell surface molecules is analyzed using quantitative PCR.

Colo-205 (American Type Culture Collection, Manassas, Va., Cat. No.CCL222) cells (1.5-2 million/condition) cultured overnight in RPMI-1640 without serum, were treated with 250-300 ng of AK155 at 37° C. for 20 min to overnight, depending on the gene to be assayed. After induction, cells were washed with phosphate buffered saline (PBS). RNA was isolated from cells by Guanidinium-Iso-Thiocyanate homogenization followed by ethanol precipitation using RNeasy® Mini Kits according to the manufacturer's instructions (Qiagen, Valencia, Calif.). 1 μg of total RNA was converted into cDNA using Superscript II® RNA H-reverse transcriptase (Gibco BRL, Rockville, Md.) according to manufacturer's instructions, with the addition of 1 μM hexamers (Promega, Madison, Wis.).

50 ng cDNA was analyzed for the expression of cytokine, chemokine or chemokine receptor genes by the fluorogenic 5′-nuclease PCR assay (Holland et al. (1991) Proc. Natl. Acad. Sci. U.S.A. 88:7276-7280) using a Perkin-Elmer ABI Prism® 7700 Sequence Detection System (ABI-PE, Foster City, Calif.). Reactions were incubated for 2 minutes at 50° C., denatured for 10 minutes at 95° C. and subjected to 40 two step amplification cycles with annealing/extension at 60° C. for 1 minute followed by denaturation at 95° C. for 15 sec. The reaction master mix was prepared according to the manufacturer's protocols to yield final concentrations of 1× PCR buffer, 200 μM dATP, dCTP, dGTP and 400 μM dUTP, 4 mM MgCl₂, 1.25 units of AmpliTaq® DNA polymerase, 0.5 units of Amp-Erase uracil-N-glycocylase, 900 nM of each primer, and 250 nM probe.

The following primers and probes were used to detect the induction of IL-8, IL-10, ICAM-1, ICAM-2 and B7-H1:

TGGCAGCCTTCCTGATTTCT (IL-8 forward) (SEQ ID NO:17)

TGCACTGACATCTAAGTTCTTTAGCA (IL-8 reverse) (SEQ ID NO:18)

TGGCAAAACTGCACCTTCACACAGAGCT (IL-8 probe) (SEQ ID NO:19)

GAGATCTCCGAGATGCCTTCA (IL-10 forward) (SEQ ID NO:20)

CAAGGACTCCTTTAACAACAAGTTGT (IL-10 reverse) (SEQ ID NO:21)

TGAAGACTTTCTTTCAAATGAAGGATCAGCTGG (IL-10 probe) (SEQ ID NO:22)

GCCAGGAGACACTGCAGACA (ICAM-1 forward) (SEQ ID NO:23)

TGGCTTCGTCAGAATCACGTT (ICAM-1 reverse) (SEQ ID NO:24)

TGACCATCTACAGCTTTCCGGCGC (ICAM-1 probe) (SEQ ID NO:25)

CGGGAAGCAGGAGTCAATGA (ICAM-2 forward) (SEQ ID NO:26)

GGGTTGCAGTGTCAGGATGA (ICAM-2 reverse) (SEQ ID NO:27)

TCAGCGTGTACCAGCCTCCAAGGC (ICAM-2 probe) (SEQ ID NO:28)

GCTGAATTGGTCATCCCAGAAC (B7-H1 forward) (SEQ ID NO:29)

GATGGCTCCCAGAATTACCAAG (B7-H1 reverse) (SEQ ID NO:30)

TCTGGCACATCCTCCAAATGAAAGGACTC (B7-H1 probe). (SEQ ID NO:31)

Amplicons were analyzed with 6-carboxy-fluorescein (FAM) labeled probes. Cytokine amplicons spanned at least one intron/exon boundary. A 18S rRNA amplicon was analyzed with a VIC® (Perkin Elmer, Foster City, Calif.) labeled probe under primer limiting conditions (Perkin-Elmer, Foster City, Calif.) and used as an internal control for quantitation of the total amount of cDNA in a multiplex reaction. Concentrations of 18S rRNA probe, forward and reverse primers were 50 nM.

AK155 up-regulated expression of IL-8, IL-10, ICAM-1, ICAM-2 and B7-H1 in Colo-205 cells. Thus, AK155 binding to a AK155 receptor complex mediates the expression of several pro-inflammatory cytokines.

### Example 3

The experiments reported in this example demonstrate that AK155 induces DNA binding of STAT3 to IFN-γ-activated-sequences (GAS) in Ba/F3 cell lines (Murine B cell line, Palacios and Steinmetz (1985) Cell 41: 727-734) transfected with the α-subunit of the AK155 receptor.

A. Preparation of Nuclear Extracts.

Mouse Pre-B cells i.e. Ba/F3, transfected with AK155 α (CPNM1) and AK155 β (IL-10Rβ) were used for electrophoretic mobility shift assays (EMSA). Nuclear extracts were prepared after stimulation of cells with AK155.

Ba/F3 transfected cells (1.5-2 million/condition) cultured overnight in RPMI-1640 without serum were treated with 250-300 ng of AK155 at 37° C. for 20 min. Cells were washed with cold PBS and resuspended in Buffer A (Basic Phosphate buffer: 50 mM HEPES, 100 mM NaF, 10 mM Na₄PPi, 2 mM Na₃V0₄, 4 mM EDTA, 2 mM sodium molybdate and protease inhibitors (Complete(Mini EDTA-Free protease inhibitor tablets; Boehringer-Mannaheim Cat No.1836170) along with 10 mM MgCl₂ and 0.2% NP40) and incubated on ice for 1 min. After centrifugation at 2000 rpm for 1 min, cells were resuspended in Buffer B (Basic phosphate buffer with protease inhibitors, 10 mM MgCl₂ and 0.25M sucrose). Lysates were spun down at 2000 rpm for 1 min and the supernatant was aspirated. 100 μl of buffer C (Basic phosphate buffer with protease inhibitors and NP40) was added and the mixture was agitated at 4° C. for 30 min. Nuclear extracts were obtained as the supernatant after centrifugation of the lysate at 15,000 rpm for 30 min at 4° C.

B. DNA Binding Assays.

Nuclear or total cellular extracts were used for DNA binding analysis. Lysates (2-3 μl) were incubated with 5 μl 2× binding buffer (20 mM Tris HCl pH 8, 200 mM KCl, 10 mM MgCl₂ and 20% Glycerol: 7 μl of 7.5% BSA, 4 μl IM DTT and 20 μl 10% NP40 2× binding buffer before use), 1 μl of poly dIdC (1 mg/ml) (Roche Molecular Biochemicals, Indianapolis, Ind., Cat. No. 1219847001) and 1 μl γATP end labeled annealed probe (the probe was gamma-activated sequence (GAS) at 10 fmol) for 30 min at room temperature. Samples were resolved on a 6% Acrylamide gel in 0.25× TBE buffer at 150V. Gels were dried and exposed to film.

The results indicated that AK155 induced DNA binding by STAT3 to gamma-interferon-activated-sequences (GAS) in Ba/F3 cells treated with AK155.

### Example 4

The experiments reported in this example demonstrate that phosphorylation of STAT3 occurs following treatment of Colo-205 cells with AK155. Colo-205 colon carcinoma cells (American Type Culture Collection, Manassas, Va., Cat. No. CCL222) were treated with prokaryotic recombinant histidine-tagged (H is-AK155) or in some cases GST-tagged (GST-AK155) AK155, and STAT3 phosphorylation was determined by phospho-STAT3-specific Western blotting.

A. Treating Colo-205 Cell Line with AK155.

Cellular extracts of Colo-205 cells were prepared to analyze phosphorylation of STAT3 after Colo-205 cells were treated with AK155. Colo-205 cells (1.5-2 million/condition) cultured overnight in RPMI-1640 without serum, were treated with prokaryotic recombinant His-AK155 (10 ng/ml) for 5, 10, 20, 30, and 60 seconds. After induction, cells were washed with phosphate buffered saline (PBS) containing protease inhibitor cocktail tablets (Complete ® Mini, EDTA-free Protease Inhibitor Cocktail Tablets, Roche Molecular Biochemicals, Cat. No. 1836170). Total cellular lysis was carried out using a Brij lysis buffer (10 mM Tris pH 7.5, 2 mM EDTA, 0.15 M NaCl, 0.875% Brij 96 and 0.125% Nonidet P40) with protease inhibitors at 4° C. Cell lysates were clarified by centrifugation at 12,000 rpm for 15 min.

B. Electrophoresis of AK155 Induced Colo-205 Cell Extracts.

Samples were prepared for SDS-PAGE analysis by adding an equal volume of sample buffer (Novex, San Diego, Calif., Cat. No. LC2676) and heating the mixture for 5 min in a boiling, water bath. Aliquots of 10-15 μg were loaded on a 10% Tris-Glycine electrophoresis system (Novex, San Diego, Calif.) and transferred to polyvinylidene difluoride (PVDF) membranes (Novex, San Diego, Calif., Cat. No. LC 2002).

C. Immunoblotting for STAT3.

After transfer, membranes were incubated in blocking solution containing 3% skim milk in Tris buffered saline solution plus Tween (TBST; 10 mM Tris-HCl pH 8.0, 0.150 mM NaCl, 0.05% Tween 20) at room temperature for 30 min. The primary antibody was anti-phospho-STAT3 (New England Biolabs, Beverly, Mass., Cat. No. 9131) Incubation with the primary antibody was carried out overnight at 4° C. at a dilution of 1:1000. Membranes were washed in TBST and incubated with an Anti Rabbit-horse radish peroxidase (HRP) conjugated secondary antibody at room temperature for 2-3 hours. HRP activity was detected using an Extended Signal chemiluminescence kit (Pierce, Rockford, Ill., Cat. No. CA47514) according to the manufacturer's recommendations. Total STAT3 in these samples was detected using a monoclonal antibody to STAT3 (BD Transduction Laboratories, Lexington, Ky., Cat. No. 21230) in a similar manner.

D. Results.

Table 1 illustrates the results of western blot analysis which showed that five minutes of treatment with AK155 was sufficient for induction of detectable levels of STAT3 phosphorylation. A lysate of the herpes virus samari-transformed T-cell line CB-15 served as a positive control. Herpes virus samurai transformed T-cells are known to secrete large amounts of AK155 (Knappe et al. (2000) J. Virol. 74:3881-3887) and are known to constitutively phosphorylate STAT3.

TABLE 1
Colo-205 cells treated with His-AK155 (10 ng/ml final).

| | | | | | | | CB |
|---|---|---|---|---|---|---|---|
| | 0 | 5 | 10 | 20 | 30 | 60 | 15 |
| | min | min | min | min | min | min | lysate |
| Phosphorylated | −−− | +++ | +++ | ++++ | ++++ | ++++ | ++ |
| STAT3 | | | | | | | |

Table 2 illustrates the results of western blot analysis that showed how the concentration of AK155 necessary for inducing STAT3 phosphorylation in Colo-205 was determined. Approximately 1 ng/ml was sufficient to induce detectable levels of STAT3 phosphorylation, whereas there was no signal at 0.3 ng/ml.

TABLE 2
Colo-205 cells
treated with His-AK155 (ng/ml final) CB-15 lysate

| | 0.0 | 0.3 | 1 | 3 | 10 | 30 | |
|---|---|---|---|---|---|---|---|
| Phosphorylated | −−− | −−− | ++ | +++ | ++++ | +++ | ++ |
| STAT3 | | | | | | | |

Table 3 illustrates the results of western blot analysis that showed that rabbit anti-AK155 antiserum (10%) reduced the STAT3 phosphorylation signal. Depending on the assay conditions (e.g., AK155 concentration), the pSTAT3 signal could even disappear completely.

TABLE 3
Western blot analysis of the Inhibition of STAT3
phosphorylation by anti-AK155 antiserum.

| | CB-15 | | HIS-AK155 + | |
|---|---|---|---|---|
| | lysate | His-AK155 | Antiserum | antiserum |
| Phosphorylated | +++++++ | +++++++ | +/− | −−−−−−−− |
| STAT3 | | | | |

AK155 is known to bind a heparin column from which it can be eluted in the early high salt (2M) fractions. Table 4 illustrates the results of western blot analysis which showed that 1 U/ml heparin was sufficient to block STAT3 phosphorylation in Colo-205 cells.

TABLE 4
Colo-205 cells
treated with His-AK155 (2 ng/ml final) CB-15 lysate.

| | 0.0 | 1 | 10 | 50 | 100 | 200 | |
|---|---|---|---|---|---|---|---|
| | units | units | units | units | units | units | |
| | heparin | heparin | heparin | heparin | heparin | heparin | |
| Phosphorylated | +++ | −−− | −−− | −−− | −−− | −−− | +++ |
| STAT3 | | | | | | | |

In summary, the experiments reported in this example show that incubation of Colo-205 cells with H is-tagged AK155 induced phosphorylation of STAT3 and that the induced phosphorylation is dependent on contact of the cells with AK155.

### Example 5

The experiments reported in this example show that only certain cell types respond to treatment with AK155 and that the cells which respond to AK155 treatment are only those that express both the α and β subunits of the AK155 receptor.

A series of cell lines in addition to Colo-205, were tested for AK155 responsiveness. Responsiveness was evaluated by measuring AK155 induced STAT3 phosphorylation as described in Example 4 above. In addition to His-AK155, different preparations of AK155 were used, namely, GST-AK155 (GST clipped off by protease treatment), AK155 purified from the Origami E. coli strain, and AK155 partially purified from the periplasm. His-AK155 had the strongest effects.

As summarized in Table 5, the cell lines HepG2 (B, E; hepatoma, IL-22 sensitive), Colo-320 (D; colon carcinoma), Molt-4 (F; lymphoma, IL-10 sensitive). Panc-1 (G; pancreatic carcinoma), HeLa (H; cervical cancer), and 293T (1; transformed embryonic kidney cells) did not react on AK155 treatment, although HepG2, Panc-1, HeLa and 293T showed some constitutive STAT3 phosphorylation. Remarkably, the supernatant from the HVS-transformed T-cell line CB-84 induced STAT3 phosphorylation in HepG2 cells which, as noted above, are not normally stimulated by AK155. The cell lines A495 (lung carcinoma, IL-22 reactive), KMH-2 (Hodgkin's disease), and human umbilical vein endothelial cells (HUVEC) did not react on AK155 treatment (data not shown).

In contrast, the human cell lines SW-403 (A; colon carcinoma), Lovo (B; colon carcinoma), and HaCaT (C; keratinoytes, known to react on IL-20) responded to AK155 treatment with STAT3 phosphorylation. This could be blocked by heparin and by a rabbit antiserum against AK155 indicating that the phosphorylation is dependent on AK155 treatment. In contrast to the pSTAT3 signals, the STAT3 control Western blots show almost constant signals.

A series of cell types was tested by RT-PCR for the transcription of members of the cytokine receptor type 2 family (IL-10R1, IL-10Rβ, AK155 α subunit (IL-20R1), IL-20R2, IL-22R, and GAPDH as positive control): Dendritic cells (DC, differentiated in vitro from monocytes), LPS-activated monocytes, SW-403 (colon carcinoma), Lovo (colon carcinoma), HepG2 (hepatoma), Colo-320 (colon carcinoma), Colo-205 (colon carcinoma), HeLa (cervical carcinoma), KMH-2 (Hodgkin's disease). Panc-1 (pancreatic carcinoma), and HaCaT (keratinocytes). The responsiveness of cells to AK155 strictly correlated with the expression of AK155 α (IL-20R) and AK155 β (IL-10R2 or IL-10Rβ); those cell types which expressed AK155 α and AK155 β responded to treatment with AK155, if both of these subunits were not present, then STAT3 phosphorylation was not observed.

TABLE 5
Phosphorylation of STAT3 in various cell types in
response to treatment with AK155.

| | | Transcription | Transcription |
|---|---|---|---|
| | STAT3 | of the | of |
| | Phosphorylation | α subunit | the β subunit |
| | in | of AK155 | of AK155 |
| | Response to | receptor | receptor |
| Cell Type | AK155 | (CPNM1) | (CPNM1) |
| Colo-205 | +++ | +++ | +++ |
| (colon carcinoma) | | | |
| HepG2 | −−−− | −−−− | +++ |
| (hepatatoma, IL22- | | | |
| sensitive) | | | |
| Colo-320 | −−−− | −−−− | +++ |
| (colon carcinoma) | | | |
| Molt-4 | −−−− | not tested | not tested |
| (lymphoma, IL-10 | | | |
| sensitive) | | | |
| Panc-1 | −−−− | −−−− | +++ |
| (pancreatic carcinoma) | | | |
| HeLa | | | |
| (cervical cancer) | −−−− | −−−− | +++ |
| 293T | | | |
| (transformed embryonic | −−−− | not tested | not tested |
| kidney) | | | |
| A495 | | | |
| (lung carcinoma, IL-22 | −−−− | not tested | not tested |
| reactive) | | | |
| KMH-2 | −−−− | −−−− | +++ |
| (Hodgkin's Disease) | | | |
| SW-403 | +++ | +++ | +++ |
| (colon carcinoma) | | | |
| Lovo | +++ | +++ | +++ |
| (colon carcinoma) | | | |
| HaCaT | +++ | +++ | +++ |
| (keratinocytes, IL-20 | | | |
| reactive) | | | |

### Example 6

The experiments reported in this example demonstrate that AK155 induces the expression of IL-8 and IL-10 in Colo-205 cells. The methods described in this example have also been used to show that anti IL-10β antibody blocks the induction of IL-8 expression which normally occurs when Colo-205 cells are treated with AK155.

A. Preparation of Substrates for ELISA.

Colo-205 cells (American Type Culture Collection, Manassas, Va., Cat. No. CCL222), at a level of 1.5-2 million/treatment condition, were cultured overnight in RPMI-1640 without serum, were aliquoted into cell culture dishes at a density of 5×10⁵/well. Cells were then treated with 200 ng of AK155 at 37° C. for 24 hours. For samples to which anti-IL-10Rβ antibody was added, the anti-IL-10Rβ antibody was added at a concentration of 10 μg/ml ½ hour prior to the addition of AK155. After AK155 treatment with or without antibody, the supernatant was collected and analyzed as described below.

B. IL-8 ELISA.

Immulon I plates were coated with Anti-IL-8 antibody (Endogen, Woburn, Mass., Cat No. M-801) in PBS at 4 μg/ml at 50 μl/well. Coated plates were incubated overnight at 4° C. or at 37° C. for 2 hours. Non-specific binding was blocked with a blocking buffer (PBS+20% fetal calf serum) and the plates were incubated at room temperature for an additional hour. Plates were washed with Wash Buffer (PBS with 0.05% Tween-20). Standards and samples were added (at 50 μl/well) in duplicates and incubated at 37° C. After two hours, plates were washed and an Anti-IL-8 biotinylated antibody (Endogen Cat. No. M-802-B) (final dilution 2 μg/ml; 50 μl/well) in conjugate buffer (Blocking buffer with 0.05% Tween 20) was added. Following an incubation of another hour at room temperature, a streptavidin-HRP linked antibody (Biosource Cat No. SNN2004). Final dilution 1:25,000 ;50 μl/well) in conjugate buffer was added. The HRP substrate—ABTS (KPL Cat. No.50-66-01) was added (100 μl/well) to develop the ELISA and the plates were read at 405 nm (Abrams (1995) Immunometric assay of mouse and human cytokines using NIP-labeled anti-cytokine antibodies, Curr. Protocols Immunol 13:61).

C. Results.

Table 6 shows that treatment with AK155 induced IL-8 and IL-10 production by Colo-205 cells.

TABLE 6
Induction of IL-8 and IL-10 by AK155.

| | 24 h | | 48 h | |
|---|---|---|---|---|
| (pg/ml) | without AK155 | with AK155 | without AK155 | with AK155 |
| IL-8 | 552 | 670 | 623 | 989 |
| IL-10 | 61 | 112 | 60 | 94 |

In addition, when anti-IL-10Rβ antibody was added to the Colo-205 cell culture 0.5 h before addition of AK155, the induction of IL-8 was blocked.

### Example 7

Class II cytokine receptors are generally heterodimers. Thus, we analyzed the expression of receptor chains that could be potential partners for IL-10R2 in cell lines that were responsive to AK155. Colo205 cells showed high levels of expression of IL-20R1 and IL-22R1, as determined by Taqman® real time quantitative polymerase chain reaction (PCR). To discriminate whether IL-20R1 or IL-22R1 were involved in the AK155 receptor, we identified other cell lines expressing one or both of these polypeptides, and tested these cell lines for responsiveness to AK155. Cell lines that were responsive to IL-22, but lacking IL-20R1 chain failed to respond to AK155. These results suggested that the receptor for AK155 is composed of IL-20R1 and IL-10R2. To examine this hypothesis, COS cells were used to reconstitute the functional receptor for AK155.

COS cells were used to reconstitute functional receptor complexes for AK155. COS cells were transfected with hIL-20R1 cDNA alone, or in combination with hIL-10R2 cDNA, and tested for their responsiveness to AK155.

AK155 induced the phosphorylation of STAT3 in COS cells transiently transfected with both receptors (IL-20R1 and IL-10R2). AK155 did not induce STAT-3 phosphorylation in non-transfected COS cells or COS cells transfected with IL-20R1 alone or IL-10R2 alone.

Expression of transfected IL-20R1 and IL-10R2 was determined by immunoprecipitation and blotting of FLAG epitope tagged receptor. FLAG-tagged IL-20R1 was immunoprecipitated with α-FLAG-M2 antibody conjugated Agarose beads (Sigma, St. Louis, Mo.). FLAG-tagged protein was detected after Western blotting using α-FLAG-M2 antibody.

Transfection was as follows. Cultures of 293T and COS cells were maintained in DMEM medium supplemented with 10% FCS, 100 U/ml penicillin, and 0.1 mg/ml streptomycin. Cells were seeded the day before transfection in 100 mm tissue culture dishes to achieve 50-75% confluence. Transient transfection was carried out using the Fugene 6® method (Roche Molecular Biochemicals, Indianapolis, Ind.). The coding sequence for IL-20R1, along with a CD8 leader sequence and an N-terminal FLAG sequence, was cloned into the pCMVSport 3 vector (Life Technologies, Rockville, Md.). The IL10R2/CRF-2-4 cDNA was cloned into a retroviral expression pMX (Misawa, et al. (2000) Proc. Nat. Acad. Sci. USA 97:3062). Equal amounts of plasmid DNA (0.003 mg each) were diluted to 0.025 ml with serum-free RPMI-1640 medium. Dilutions of Fugene 6t (Roche Molecular Biochemicals, Indianapolis, Ind.) (maintaining a 0.002 ml Fugene6®:0.001 mg DNA ratio) in serum-free medium were added to DNA. After 15 min incubation at room temperature, the mixture was dispersed dropwise into the cultures. Transfected cells were usually harvested after two days of incubation.

STAT3 phosphorylation was determined as follows. Transfected COS cells were serum starved overnight before use, in RPMI-1640, for STAT3 phosphorylation experiments. Cells were washed twice with serum-free RPMI-1640. From 2.0-2.5 million cells were used per test condition. A positive signal for STAT3 phosphorylation was generated by treatment with hIFN-α (0.1 μg/ml). Purified histidine-tagged AK155 (0.5 μg/ml) was used to induce STAT3 phosphorylation, where incubations were for 20 min at 37° C. Cells were then lysed and protein were separated by SDS PAGE, with analysis by Western blotting with anti-STAT3-phosphage antibodies.

Cell lysis was carried out as follows. Cells were washed with PBS containing protease inhibitor tablets (complete mini EDTA-free, Boehringer-Mannheim, Cat. No. 1836170). Total cellular lysis was by exposure to Brij buffer (10 mM Tris, pH 7.5, 2 mM EDTA, 0.15 M NaCl, 0.875% Brij 96, and 0.125% Nonidet P40), with protease inhibitors and phosphatase inhibitors, at 4° C. The phosphatase inhibitors were sodium fluoride (10 mM) and sodium vanadate (10 mM). Cell lysates were clarified by centrifugation (12,000 rpm, 15 min). Samples were prepared for SDS PAGE analysis by adding an equal volume of sample buffer (Novex, San Diego, Calif., Cat. No. LV2676) and heating the mixture for 5 min in a boiling water bath. Aliquots (25-30 μl) were loaded on a 10% Tris-Glycine electrophoretic gel (Novex, San Diego, Calif.) and transferred to polyvinylidene difluoride (PVDF) membranes (Novex, San Diego, Calif., Cat. No. LC2002).

After transfer, membranes were incubated in blocking solution containing 3% skimmed milk in Tris buffered saline solution (10 mM Tris HCl, pH 8.0, 150 mM NaCl, 0.05% Tween 20) for 30 min at room temperature. Primary antibody-anti-phospho-STAT3 (New England Biolabs, cat. no. 9131) was diluted 1:1000, added to membranes, and incubated overnight at 4° C. Membranes were washed in the Tris buffered saline solution, and incubated with anti-rabbit-horse radish peroxidase conjugated secondary antibody, where incubation was for 2-3 h at room temperature. Horse radish peroxidase activity was detected using the Extended Signal® chemiluminescence kit (Pierce, Rockford, Ill., Cat. No. CA47514). Total STAT3 was detected using a monoclonal antibody to STAT3 (BD Transduction Laboratories, Lexington, Ky., Cat. No. 21320).

### Example 8

Control studies involved exposure to interferon-α (IFN-α) or interleukin-10 (IL-10). Activity of these cytokines was assessed by measuring STAT3 phosphorylation. Cells (2.0-2.5 million cells/test) were incubated in serum-free RPMI-1640 overnight and induced with IFN-α (200 ng/ml), IL-10 (200 ng/ml), or AK155 (500 ng/ml) at the indicated concentration for 20 min at 37° C.

As a control, STAT3 was tested for its ability to be phosphorylated in response to IFN-α. Analysis by Western blotting demonstrated that non-transfected cells and cells transfected with various receptor chains all showed strong STAT-3 phosphorylation in response to IFN-α. Reblotting with reagents specific to STAT-3 indicated that equal amounts of STAT-3 were present in all samples.

As an additional control, STAT3 was tested for its ability to be phosphorylated in response to IL-10. Human IL-10 utilizes a heterodimeric receptor composed of IL-10R1 and IL-10R2. A functional receptor for human IL-10 was reconstituted in COS cells transiently transfected with human IL-10R1 alone. This hIL-10R1 polypeptide was able to utilize the endogenous monkey IL-10R2 polypeptide (in the COS cells), as revealed by the induction of STAT3 phosphorylation in response to IL-10. Non-transfected COS cells did not respond to either IL-10 or to AK155.

Many modifications and variations of this invention, as will be apparent to one of ordinary skill in the art can be made to adapt to a particular situation, material, composition of matter, process, process step or steps, to preserve the objective, spirit and scope of the invention. All such modifications are intended to be within the scope of the claims appended hereto without departing from the spirit and scope of the invention. The specific embodiments described herein are offered by way of example only, and the invention is to be limited by the terms of the appended claims, along with the full scope of equivalents to which such claims are entitled; and the invention is not to be limited by the specific embodiments that have been presented herein by way of example.

What is claimed is:

1. A cell recombinantly altered to express an exogenous AK155 cytokine receptor comprised of α and β subunits wherein:
i) the amino acid sequence of the AK155 receptor subunit α is at least 75% identical to SEQ ID NO: 1; and,
ii) the amino acid sequence of the AK155 receptor subunit β is at least 75% identical to SEQ ID NO:2; and,
the cytokine receptor, when expressed in Ba/F3 cells, binds to AK155 and stimulates binding of STAT3 to IFN gamma-activated-sequences.

2. The cytokine receptor of claim 1, wherein the α subunit is SEQ ID NO:1.

3. The cytokine receptor of claim 1, wherein the β subunit is SEQ ID NO:2.

4. A method for identifying anti-inflammatory agents wherein the agent inhibits AK155 activation of an AK155 receptor, which is defined as having an amino acid sequence of its α subunit at least 75% homologous to SEQ ID NO:1; and the amino acid sequence of its β subunit at least 75% homologous to SEQ ID NO:2, the method comprising:
i) contacting AK155 in a solution containing the receptor complex and a compound suspected of inhibiting receptor activation induced by binding of AK155 to the receptor; and,
ii) detecting an inhibition of AK155 receptor activation that results from AK155 binding to the AK155 receptor.

5. The method of claim 4 wherein the receptor is the AK155 receptor.

6. The method of claim 4 wherein the receptor is expressed in a cell.

7. The method of claim 4 wherein the inhibitor is a ligand that is a competitive inhibitor of AK155 binding to its receptor.

8. The method of claim 4 wherein the inhibitor is a receptor specific antibody.

9. The method of claim 4 wherein the inhibitor is an antibody which binds to AK155.

10. The method of claim 4 wherein detection is by analysis of the expression of IL-8.

11. The method of claim 4 wherein detection is by analysis of the expression of ICAM-1. analysis of the expression of ICAM-2.

12. The method of claim 4 wherein detection is by analysis of the expression of B7-H1.

13. The method of claim 4 wherein detection is by analysis of STAT3 translocation to the nucleus of a cell.

14. The method of claim 4 wherein detection is by analysis of STAT3 phosphorylation.

15. The method of claim 4 wherein detection is by analysis of binding of activated STAT3 or STAT3-receptor complex to DNA at IFN gamma-activated-sequences.

16. A method of inhibiting inflammation in a patient suffering from inflammatory disease, the method comprising the administration of an antagonist of the AK155-AK155-receptor complex in an amount effective to inhibit AK155 activated inflammation.

17. The method of claim 16 wherein the antagonist is an AK155 receptor antibody.

18. The method of claim 16 wherein the antagonist is an antibody which binds to AK155.

19. The method of claim 16 wherein the antagonist is a ligand that is a competitive inhibitor of AK155 binding to its receptor.

20. A method for detecting binding of AK155 to cell surface glycosaminoglycans of a cell comprising adding AK155 to a first cell, adding AK155 and heparin to a second cell, and comparing binding of AK155 to said first and second cells, wherein lower binding with added heparin signifies that binding of AK155 to cell surface glycosaminoglycans occurs without added heparin.


SEQUENCE LISTING (31 sequences)

SEQ ID NO: 1 - protein, 553 aa, Homo sapiens [sequence omitted]

SEQ ID NO: 2 - protein, 273 aa, Homo sapiens
MAWSLGSWLGGCLLVSALGMVPPPENVRMNSVNFKNILQWESPAFAKGNLTFTAQYLSYRIFQDKCMNTTLTECDFSSLSKYGDHTLRVRAEFADEHSDWVNITFCPVDDTIIGPPGMQVEVLDDSLHMRFLAPKIENEYETWTMKNVYNSWTYNVQYWKNGTDEKFQITPQYDFEVLRNLEPWTTYCVQVRGFLPDRNKAGEWSEPVCEQTTHDETVPSWMVAVILMASVFMVCLALLGCFSLLWCVYKKTKYAFSPRNSLPQHLKEVGRME

SEQ ID NO: 3 - DNA, 26 bp, Artificial Sequence
  Forward primer alpha subunit of AK155 receptor.
atgaagaatgtcctacaatggactcc

SEQ ID NO: 4 - DNA, 26 bp, Artificial Sequence
  Reverse primer alpha subunit AK155 receptor.
tcagttttccatctgcacatataacc

SEQ ID NO: 5 - DNA, 19 bp, Artificial Sequence
  Forward primer beta subunit AK155 receptor.
atggcgtggagtcttggga

SEQ ID NO: 6 - DNA, 26 bp, Artificial Sequence
  Reverse primer beta subunit AK155 receptor.
tcactccatcctacctacctctttca

SEQ ID NO: 7 - DNA, 3485 bp, Homo sapiens
tccagctgggtagccgggggagcgcgcgtgggggctccgcgagtcgctcgcccttggtttctggggaagcctgggggacgcggctgtggcggaggcgccctgggactcaggtcgcctggagcgtggcacgcagagccccaggcgcggagctgaggccgcgcggccgcgcttggccccagcgggcgtgggactgagcagtctgctgccccccgacatgtgacccagccccgccgcccatgcgggctcccggccgcccggccctgcggccgctgccgctgccgccgctgctgctgttgctcctggcggcgccttggggacgggcagttccctgtgtctctggtggtttgcctaaacctgcaaacatcaccttcttatccatcaacatgaagaatgtcctacaatggactccaccagagggtcttcaaggagttaaagttacttacactgtgcagtatttcatatatgggcaaaagaaatggctgaataaatcagaatgcagaaatatcaatagaacctactgtgatctttctgctgaaacttctgactacgaacaccagtattatgccaaagttaaggccatttggggaacaaagtgttccaaatgggctgaaagtggacggttctatccttttttagaaacacaaattggcccaccagaggtggcactgactacagatgagaagtccatttctgttgtcctgacagctccagagaagtggaagagaaatccagaagaccttcctgtttccatgcaacaaatatactccaatctgaagtataacgtgtctgtgttgaatactaaatcaaacagaacgtggtcccagtgtgtgaccaaccacacgctggtgctcacctggctggagccgaacactctttactgcgtacacgtggagtccttcgtcccagggccccctcgccgtgctcagccttctgagaagcagtgtgccaggactttgaaagatcaatcatcagagttcaaggctaaaatcatcttctggtatgttttgcccatatctattaccgtgtttcttttttctgtgatgggctattccatctaccgatatatccacgttggcaaagagaaacacccagcaaatttgattttgatttatggaaatgaatttgacaaaagattctttgtgcctgctgaaaaaatcgtgattaactttatcaccctcaatatctcggatgattctaaaatttctcatcaggatatgagtttactgggaaaaagcagtgatgtatccagccttaatgatcctcagcccagcgggaacctgaggccccctcaggaggaagaggaggtgaaacatttagggtatgcttcgcatttgatggaaattttttgtgactctgaagaaaacacggaaggtacttctttcacccagcaagagtccctcagcagaacaatacccccggataaaacagtcattgaatatgaatatgatgtcagaaccactgacatttgtgcggggcctgaagagcaggagctcagtttgcaggaggaggtgtccacacaaggaacattattggagtcgcaggcagcgttggcagtcttgggcccgcaaacgttacagtactcatacacccctcagctccaagacttagaccccctggcgcaggagcacacagactcggaggaggggccggaggaagagccatcgacgaccctggtcgactgggatccccaaactggcaggctgtgtattccttcgctgtccagcttcgaccaggattcagagggctgcgagccttctgagggggatgggctcggagaggagggtcttctatctagactctatgaggagccggctccagacaggccaccaggagaaaatgaaacctatctcatgcaattcatggaggaatgggggttatatgtgcagatggaaaactgatgccaacacttccttttgccttttgtttcctgtgcaaacaagtgagtcacccctttgatcccagccataaagtacctgggatgaaagaagttttttccagtttgtcagtgtctgtgagaattacttatttcttttctctattctcatagcacgtgtgtgattggttcatgcatgtaggtctcttaacaatgatggtgggcctctggagtccaggggctggccggttgttctatgcagagaaagcagtcaataaatgtttgccagactgggtgcagaatttattcaggtgggtgtactctggcctcttggttcattattttcaaacaagcacacttgtacaattattttctgggtacttcccatatgcacatagcactgtaaaaaatatttcccaaagatcactcattttataaataccactttttcagaattgggtttattgcgagcaggaggagatacttaaaacatgcacatataccaggttggtggtaagttggtcacatgtgaaaacctcaactatttaatcatcatgattcatattttgagtgaatacatcaggcacagaccttcatgatatcacacactcttggctactttaagaggccatctttaatactttatgagtagttctggagtgtaaacataaacgagtattcttttgtagtcagaaaagtgtcctctcaataatttagtaggggcttattgtctctcaaaactaacctaaaagaaaatgacacattttataatagaatattacatttatttctggaagtgtgttttcaaaaagatatttacatagtctgtaaactagaaagtgttaggtaaagctctaggttactgtgttactattataatattaaacattcgaataggcagtcgttcaaagactctttggaatatctatgaatgaatatcctctattcttataatattaaaacccataagtaaatataggacatacaagagaaatgagttaaatgactatgtaagggagagtttattaaaatttgatgaaatttactgtaggaactaaactatgccataaaacaatagctttctagttcatttccagtaactgttcccatctcctttaccacttgttaagaaaattaaattcttcagtcacgctgctttaaaatgggacaaaatctattaagttgaaccatatataattgtggatatttggctgtttttaatctgacaagcagtaacttcatatggtttgccttaatatatatttgttttagtcatgaactcataatccattgatgctctttcatgagaagagatatgacccatatttccttattgatattattggtacaggcagacaaccctggtaggagagatggattctggggtcatgacctttcgtgattatccgcaaatgcaaacagtttcagatctaatggtttaatttagggagtaattatattaatcagagtgttctgttattctcaatctttatagaaacgattctgctggttttgaagaacagatgtattacactaactgtaaaagtagttcaagagtgagaaagaataaattgttattaagagcaaaagaaaaataaagtgattgatgata

SEQ ID NO: 8 - DNA, 822 bp, Homo sapiens
atggcgtggagtcttgggagctggctgggtggctgcctgctggtgtcagcattgggaatggtaccacctcccgaaaatgtcagaatgaattctgttaatttcaagaacattctacagtgggagtcacctgcttttgccaaagggaacctgactttcacagctcagtacctaagttataggatattccaagataaatgcatgaatactaccttgacggaatgtgatttctcaagtctttccaagtatggtgaccacaccttgagagtcagggctgaatttgcagatgagcattcagactgggtaaacatcaccttctgtcctgtggatgacaccattattggaccccctggaatgcaagtagaagtacttgatgattctttacatatgcgtttcttagcccctaaaattgagaatgaatacgaaacttggactatgaagaatgtgtataactcatggacttataatgtgcaatactggaaaaacggtactgatgaaaagtttcaaattactccccagtatgactttgaggtcctcagaaacctggagccatggacaacttattgtgttcaagttcgagggtttcttcctgatcggaacaaagctggggaatggagtgagcctgtctgtgagcaaacaacccatgacgaaacggtcccctcctggatggtggccgtcatcctcatggcctcggtcttcatggtctgcctggcactcctcggctgcttctccttgctgtggtgcgtttacaagaagacaaagtacgccttctcccctaggaattctcttccacagcacctgaaagaggtaggtaggatggagtga

SEQ ID NO: 9 - protein, 171 aa, Homo sapiens
MLVNFILRCGLLLVTLSLAIAKHKQSSFTKSCYPRGTLSQAVDALYIKAAWLKATIPEDRIKNIRLLKKKTKKQFMKNCQFQEQLLSFFMEDVFGQLQLQGCKKIRFVEDFHSLRQKLSHCISCASSAREMKSITRMKRIFYRIGNKGIYKAISELDILLSWIKKLLESSQ

SEQ ID NO: 10 - DNA, 1047 bp, Homo sapiens
ctgtgagtgacacacgctgagtggggtgaagggaaatgctggtgaatttcattttgaggtgtgggttgctgttagtcactctgtctcttgccattgccaagcacaagcaatcttccttcaccaaaagttgttacccaaggggaacattgtcccaagctgttgacgctctctatatcaaagcagcatggctcaaagcaacgattccagaagaccgcataaaaaatatacgattattaaaaaagaaaacaaaaaagcagtttatgaaaaactgtcaatttcaagaacagcttctgtccttcttcatggaagacgtttttggtcaactgcaattgcaaggctgcaagaaaatacgctttgtggaggactttcatagccttaggcagaaattgagccactgtatttcctgtgcttcatcagctagagagatgaaatccattaccaggatgaaaagaatattttataggattggaaacaaaggaatctacaaagccatcagtgaactggatattcttctttcctggattaaaaaattattggaaagcagtcagtaaaccaaagccaagtacattgattttacagttattttgaaatacaataagaactgctagaaatatgtttataacagtctatttcttttaaaaactttttaacataatactgacggcatgttaggtgattcagaatagacaagaaggatttagtaaattaacgttttggatataagttgtcactaatttgcacattttctgtgttttcaaataatgtttccattctgaacatgttttgtcattcacaagtacattgtgtcaacttaatttaaagtatgtaacctgaattaactcgtgtaatatttgtgtgtggagtgggatgtggggggtggagggggaatgacagatttctggaatgcaatgtaatgttactgagacttaaatagatgttatgtatatgattgtctgtttaagtgtttgaaaattgttaattatgcccagtgtgaacttagtacttaacacattttgattttaattaaataaattgggtttccttctc

SEQ ID NO: 11 - protein, 553 aa, Homo sapiens
MRAPGRPALRPLPLPPLLLLLLAAPWGRAVPCVSGGLPKPANITFLSINMKNVLQWTPPEGLQGVKVTYTVQYFIYGQKKWLNKSECRNINRTYCDLSAETSDYEHQFYAKVKAIWGTKCSKWAESGRFYPFLETQIGPPEVALTTDEKSISVVLTAPEKWKRNPEDLPVSMQQIYSNLKYNVSVLNTKSNRTWSQCVTNHTLVLTWLEPNTLYCVHVESFVPGPPRRAQPSEKQCARTLKDQSSEFKAKIIFWYVLPISITVFLFSVMGYSIYRYIHVGKEKHPANLILIYGNEFDKRFFVPAEKIVINFITLNISDDSKISHQDMSLLGKSSDVSSLNDPQPSGNLRPPQEEEEVKHLGYASHLMEIFCDSEENTEGTSFTQQESLSRTIPPDKTVIEYEYDVRTTDICAGPEEQELSLQEEVSTQGTLLESQAALAVLGPQTLQYSYTPQLQDLDPLAQEHTDSEEGPEEEPSTTLVDWDPQTGRLCIPSLSSFDQDSEGCEPSEGDGLGEEGLLSRLYEEPAPDRPPGENETYLMQFMEEWGLYVQMEN

SEQ ID NO: 12 - protein, 553 aa, Homo sapiens
MRAPGRPALRPLPLPPLLLLLLAAPWGRAVPCVSGGLPKPANITFLSINMKNVLQWTPPEGLQGVKVTYTVQYFIYGQKKWLNKSECRNINRTYCDLSAETSDYEHQYYAKVKAIWGTKCSKWAESGRFYPFLETQIGPPEVALTTDEKSISVVLTAPEKWKRNPQDLPVSMQQIYSNLKYNVSVLNTKSNRTWSQCVTNHTLVLTWLEPNTLYCVHVESFVPGPPRRAQPSEKQCARTLKDQSSEFKAKIIFWYVLPISITVFLFSVMGYSIYRYIHVGKEKHPANLILIYGNEFDKRFFVPAEKIVINFITLNISDDSKISHQDMSLLGKSSDVSSLNDPQPSGNLRPPQEEEEVKHLGYASHLMEIFCDSEENTEGTSFTQQESLSRTIPPDKTVIEYEYDVRTTDICAGPEEQELSLQEEVSTQGTLLESQAALAVLGPQTLQYSYTPQLQDLDPLAQEHTDSEEGPEEEPSTTLVDWDPQTGRLCIPSLSSFDQDSEGCEPSEGDGLGEEGLLSRLYEEPAPDRPPGENETYLMQFMEEWGLYVQMEN

SEQ ID NO: 13 - protein, 553 aa, Homo sapiens
MRAPGRPALRPLPLPPLLLLLLAAPWGRAVPCVSGGLPKPANITFLSINMKNVLQWTPPEGLQGVKVTYTVQYFIYGQKKWLNKSECRNINRTYCDLSAETSDYEHQTYAKVKAIWGTKCSKWAESGRFYPFLETQIGPPEVALTTDEKSISVVLTAPEKWKRNPEDLPVSMQQVYSNLKYNVSVLNTKSNRTWSQCVTNHTLVLTWLEPNTLYCVHVESFVPGPPRRAQPSEKQCARTLKDQSSEFKAKIIFWYVLPISITVFLFSVMGYSIYRYIHVGKEKHPANLILIYGNEFDKRFFVPAEKIVINFITLNISDDSKISHQDMSLLGKSSDVSSLNDPQPSGNLRPPQEEEEVKHLGYASHLMEIFCDSEENTEGTSFTQQESLSRTIPPDKTVIEYEYDVRTTDICAGPEEQELSLQEEVSTQGTLLESQAALAVLGPQTLQYSYTPQLQDLDPLAQEHTDSEEGPEEEPSTTLVDWDPQTGRLCIPSLSSFDQDSEGCEPSEGDGLGEEGLLSRLYEEPAPDRPPGENETYLMQFMEEWGLYVQMEN

SEQ ID NO: 14 - protein, 273 aa, Homo sapiens
MAWSLGSWLGGCLLVSALGMVPPPENVRMNSVNFKNILQWESPAFAKGNLTFTAQYLSYRIFQDKCMNTTLTECDFSSLSKYGDHTLRVRAEFADEHSDWVNITFCPVDDTIIGPPGMQVEVLDNSLHMRFLAPKIENEYETWTMKNVYNSWTYNVQYWKNGTDEKFQITPQYDFEVLRNLEPWTTYCVQVRGFLPDRNKAGEWSEPVCEQTTHDETVPSWMVAVILMASVFMVCLALLGCFSLLWCVYKKTKYAFSPRNSLPQHLKEVGRME

SEQ ID NO: 15 - protein, 273 aa, Homo sapiens
MAWSLGSWLGGCLLVSALGMVPPPENVRMNSVNFKNILQWESPAFAKGNLTFTAQYLSYRIFQDKCMNTTLTECDFSSLSKYGDHTLRVRAEFADEHSDWVNITFCPVDDTIIGPPGMQVEVLDDSLHMRFLAPKIENEYETWTMKNVYNSWTYNVQYWKNGSDEKFQITPQYDFEVLRNLEPWTTYCVQVRGFLPDRNKAGEWSEPVCEQTTHDETVPSWMVAVILMASVFMVCLALLGCFSLLWCVYKKTKYAFSPRNSLPQHLKEVGRME

SEQ ID NO: 16 - protein, 273 aa, Homo sapiens
MAWSLGSWLGGCLLVSALGMVPPPENVRMNSVNFKNILQWESPAFAKGNLTFTAQYLSYRIFQDKCMNTTLTECDFSSLSKYGDHTLRVRAEFADEHSDWVNITFCPVDDTIIGPPGMQVEVLDDSLHMRFLAPKIENEYETWTMKNVYNSWTYNVQYWKNGTDEKFQVTPQYDFEVLRNLEPWTTYCVQVRGFLPDRNKAGEWSEPVCEQTTHDETVPSWMVAVILMASVFMVCLALLGCFSLLWCVYKKTKYAFSPRNSLPQHLKEVGRME

SEQ ID NO: 17 - DNA, 20 bp, Artificial Sequence
  Forward primer IL-8.
tggcagccttcctgatttct

SEQ ID NO: 18 - DNA, 26 bp, Artificial Sequence
  Reverse primer IL-8.
tgcactgacatctaagttctttagca

SEQ ID NO: 19 - DNA, 28 bp, Artificial Sequence
  Probe for IL-8.
tggcaaaactgcaccttcacacagagct

SEQ ID NO: 20 - DNA, 21 bp, Artificial Sequence
  Forward primer IL-10.
gagatctccgagatgccttca

SEQ ID NO: 21 - DNA, 26 bp, Artificial Sequence
  Reverse primer for IL-10.
caaggactcctttaacaacaagttgt

SEQ ID NO: 22 - DNA, 33 bp, Artificial Sequence
  Probe for IL-10.
tgaagactttctttcaaatgaaggatcagctgg

SEQ ID NO: 23 - DNA, 20 bp, Artificial Sequence
  Forward primer for ICAM-1.
gccaggagacactgcagaca

SEQ ID NO: 24 - DNA, 21 bp, Artificial Sequence
  Reverse primer for ICAM-1.
tggcttcgtcagaatcacgtt

SEQ ID NO: 25 - DNA, 24 bp, Artificial Sequence
  Probe for ICAM-1.
tgaccatctacagctttccggcgc

SEQ ID NO: 26 - DNA, 20 bp, Artificial Sequence
  Forward primer for ICAM-2.
cgggaagcaggagtcaatga

SEQ ID NO: 27 - DNA, 20 bp, Artificial Sequence
  Reverse primer for ICAM-2.
gggttgcagtgtcaggatga

SEQ ID NO: 28 - DNA, 24 bp, Artificial Sequence
  Probe for ICAM-2.
tcagcgtgtaccagcctccaaggc

SEQ ID NO: 29 - DNA, 22 bp, Artificial Sequence
  Forward primer for B7-H1.
gctgaattggtcatcccagaac

SEQ ID NO: 30 - DNA, 22 bp, Artificial Sequence
  Reverse primer for B7-H1.
gatggctcccagaattaccaag

SEQ ID NO: 31 - DNA, 29 bp, Artificial Sequence
  Probe for B7-H1.
tctggcacatcctccaaatgaaaggactc